(19) United States
(12) Patent Application Publication
(10) Pub. No.: US 2025/0102182 A1
(43) Pub. Date: Mar. 27, 2025

(54) Air Purifier

(71) Applicants: CRAWFORD GLOBAL LIMITED, Hong Kong (CN); ETI SOLID STATE LIGHTING INC, Wheeling, IL (US); DERNIER AND HAMLYN INC., Wheeling, IL (US)

(72) Inventors: Donglei WANG, Zhuhai, Guangdong (CN); Jianwei CHEN, Zhuhai, Guangdong (CN); Qunyong CAO, Zhuhai, Guangdong (CN); Jingmei HUANG, Zhuhai, Guangdong (CN)

(21) Appl. No.: 18/726,103

(22) PCT Filed: Dec. 28, 2022
(86) PCT No.: PCT/CN2022/143051

§ 371 Date: Jul. 1, 2024

(30) Foreign Application Priority Data
Dec. 30, 2021 (CN) 202111661936.5
Aug. 26, 2022 (CN) 202222270737.8

(51) Int. Cl. F24F 13/02; F24F 8/108; F24F 8/22; F24F 8/30

(52) U.S. Cl. CPC F24F 13/02; F24F 8/108; F24F 8/22; F24F 8/30

(57) ABSTRACT

Provided is an air purifier. The air purifier includes a casing, an air inlet, a first air outlet and a second air outlet, wherein air ducts are arranged in the casing, the air inlet, the first air outlet and the second air outlet are provided on the casing, a first air duct of the air ducts is arranged between the air inlet and the first air outlet, a second air duct of the air ducts is arranged between the air inlet and the second air outlet, the first air duct and the second air duct are arranged in the casing, a first fan is arranged in the first air duct, and a second fan is arranged in the second air duct. The double air ducts are arranged in the casing, the first fan is arranged in the first air duct, the second fan is arranged in the second air duct.

### CROSS-REFERENCE TO RELATED APPLICATION

The disclosure claims the benefit of priority to patent application No. 202111661936.5, filed with the China National Intellectual Property Administration on Dec. 30, 2021 and entitled “Double-air-duct assembly and air-purifying hand dryer”. Moreover, the disclosure claims the benefit of priority to patent application No. 202222270737.8, filed with the China National Intellectual Property Administration on Aug. 26, 2022 and entitled “Air purifier”.

### TECHNICAL FIELD

The disclosure relates to the field of air purification, and particularly relates to an air purifier.

### BACKGROUND

With the demand for air quality increasing, not only fresh and clean air, but also healthy and sterile air are required. In a public toilet, bacteria or viruses are extremely likely to gather in the air due to large flow of people, and an unpleasant odor is likely to be produced in the toilet, causing instinctive antipathy and severe discomfort of people. However, an existing hand dryer in the toilet, single in function, can only dry hands. An existing hand dryer having an air purifying function is equipped with only one fan. The fan is required to be switched between a high-speed air supplying function and a low-speed plasma air purifying function within a long time. As a result, wear and tear as well as power consumption of the fan is heavy, the service life of the fan is shortened, and the requirement of long-term performance cannot be satisfied.

When an air purifier is applied to a toilet, a fragrance diffuser is arranged in the air purifier for a higher comfort level in the toilet, and a fragrance released from the fragrance diffuser is discharged out of the air purifier along with the air. As a consumable, the fragrance diffuser will be used up after a period of time. Then, it is required to add a new fragrance diffuser to an air duct. However, since a purifying assembly in an existing air purifier is mounted in a closed manner in the air duct, if a new fragrance diffuser is to be added, it is required to dismount components of the air duct, open the air duct and then mount the new fragrance diffuser in the air duct. Such a process is laborious.

### SUMMARY

A main objective of the disclosure is to provide an air purifier which can reduce wear and tear of fans. In addition, another objective of the disclosure is to achieve an air purifier having a fragrance diffuser rapid to add and a purifying assembly rapid to replace.

In order to realize the above main objective, the disclosure provides an air purifier. The air purifier includes a casing, an air inlet, a first air outlet and a second air outlet, where air ducts are arranged in the casing, the air inlet, the first air outlet and the second air outlet are provided on the casing, a first air duct of the air ducts is arranged between the air inlet and the first air outlet, a second air duct of the air ducts is arranged between the air inlet and the second air outlet, the first air duct and the second air duct are arranged in the casing, a first fan is arranged in the first air duct, and a second fan is arranged in the second air duct.

From the above solution, it can be seen that the double air ducts are arranged in the casing, the first fan is arranged in the first air duct, and the second fan is arranged in the second air duct. The first fan and the second fan have different air volumes, the fan having a greater air volume is mainly applied to perform a hand drying function, and the fan having a less air volume is applied to perform an air purifying function. The fan having a high-speed air supplying function and the fan having a low-speed air purifying function are independent from each other, such that it is not required to frequently switch modes of the fans, excessive wear and tear of the fans are avoided, and service life of the fans is prolonged.

In an embodiment, the air purifier further includes at least one of a functional purifying member and a fragrance diffuser, where at least one pluggable opening is provided on the casing, and the functional purifying member and/or the fragrance diffuser passes through the at least one pluggable opening so as to be mounted in the air duct by means of a limiting structure.

From the above solution, it can be seen that the functional purifying member and the fragrance diffuser are arranged in the air duct through the pluggable opening, such that the fragrance diffuser and the functional purifying member are mounted in the air duct in an open manner. The fragrance diffuser and the functional purifying member are mounted or dismounted in a pluggable manner such that the fragrance diffuser and the functional purifying member can be rapidly replaced.

In an embodiment, the functional purifying member includes at least one of a primary-efficiency filter screen, a high-efficiency particulate air (HEPA) filter screen and an ozone removal screen.

It can be seen that the primary-efficiency filter screen, the HEPA filter screen and the ozone removal screen are all non-electrified functional purifying members. Technical requirements of mounting such non-electrified functional purifying members through the pluggable opening are relatively low, and a user can more rapidly and simply mount such non-electrified functional purifying members through the pluggable opening.

In an embodiment, the limiting structure includes a limiting plate, the limiting plate is connected to the functional purifying member, the limiting plate is located outside the pluggable opening, and the casing supports the limiting plate; and/or
the functional purifying member is in interference fit with the pluggable opening; and/or
the limiting structure includes at least two limiting convex ribs, the at least two limiting convex ribs are arranged on an inner side wall of the air duct, the at least two limiting convex ribs are arranged opposite each other, and the functional purifying member is located between the at least two limiting convex ribs; and/or
the limiting structure is a limiting groove, and the limiting groove is formed on the inner side wall of the air duct in an outward protrusion manner.

It can be seen that the functional purifying member is fixed in the air duct through the pluggable opening in a plurality of manners, such as a manner that the casing supports the limiting plate connected to the functional purifying member, a manner of interference fit, a manner of being limited between the two limiting convex ribs, and a manner of providing the limiting groove.

In an embodiment, the limiting plate is adapted to the pluggable opening, a first grabbing recess is arranged on the casing, the first grabbing recess is in communication with the pluggable opening, and the limiting plate is located above the first grabbing recess; or a second grabbing recess and a third grabbing recess are arranged on the casing, the pluggable opening is located in the second grabbing recess and the third grabbing recess, and the second grabbing recess and the third grabbing recess are in communication with the pluggable opening.

It can be seen that in a case that the functional purifying member is arranged in the air duct by means of the limiting plate, the pluggable opening limits the limiting plate, and the limiting plate in the pluggable opening can be better taken out by means of the first grabbing recess. In a case that the functional purifying member is in interference fit with the pluggable opening, the clamped functional purifying member can be more conveniently taken out by means of the grabbing recesses on two sides of the pluggable opening.

In an embodiment, the limiting structure includes a mounting frame, the mounting frame is hung in the pluggable opening, and the fragrance diffuser is arranged on the mounting frame.

It can be seen that the mounting frame serves as a mounting carrier of the fragrance diffuser, and the fragrance diffuser is arranged in the air duct in a manner that the mounting frame is hung in the pluggable opening.

In an embodiment, the mounting frame includes a plurality of frameworks, the plurality of frameworks are cross-connected to each other, at least one limiting concave portion is formed between two adjacent frameworks, and the fragrance diffuser passes through a concave opening of the limiting concave portion so as to be snap-fitted in the limiting concave portion.

It can be seen that the mounting frame is formed by splicing a plurality of frameworks, and the fragrance diffuser is arranged on the mounting frame by means of the limiting concave portion. The fragrance diffuser is fixed through snap-fit by means of the limiting concave portion on the mounting frame, such that the situation that since the fragrance diffuser is excessively blocked, flow of the air in the air duct is affected is avoided.

In an embodiment, limiting blocks are arranged on sides, facing each other, of two adjacent frameworks respectively, the limiting concave portions are arranged on the two limiting blocks respectively, and concave openings of the two limiting concave portions have the same opening direction; and openings are provided on sides, facing each other, of the two limiting blocks respectively, and the fragrance diffuser passes through the two openings so as to be clamped between the two limiting blocks.

It can be seen that the fragrance diffuser is clamped on the mounting frame by means of the two limiting blocks, such that stability of the fragrance diffuser on the mounting frame is better.

In an embodiment, the limiting structure includes a limiting chamber, the limiting chamber is located in the air duct, the fragrance diffuser sequentially passes through the pluggable opening and a first opening of the limiting chamber so as to enter the limiting chamber, an air hole is provided on a side wall of the limiting chamber, and the air duct passes through the air hole.

It can be seen that the fragrance diffuser is placed in the limiting chamber, such that the fragrance diffuser is placed in the air duct. Moreover, the air duct passes through the air hole on the limiting chamber, such that the limiting chamber is prevented from affecting flow of the air in the air duct.

In an embodiment, the limiting chamber includes a supporting plate and a baffle, the baffle is connected to the supporting plate, the baffle delimits the limiting chamber in a circumferential direction of the supporting plate, and the air hole is provided on the baffle.

It can be seen that the supporting plate serves as a bottom of the limiting chamber, the baffle serves as a side wall of the limiting chamber, the supporting plate supports the fragrance diffuser, and the baffle delimits the limiting chamber in the circumferential direction of the supporting plate. Therefore, the limiting chamber is formed by a simplest structure.

In an embodiment, the fragrance diffuser is arranged in the air duct by means of a mounting frame, a second opening is provided on the limiting chamber and faces the mounting frame, the air duct passes through the second opening, and the fragrance diffuser is located at the second opening and is clamped between a side wall of the limiting chamber and the mounting frame.

It can be seen that through the second opening, the fragrance diffuser passes through the first opening of the limiting chamber, enters the limiting chamber, and is clamped between the baffle and the mounting frame. Therefore, in the air duct, limiting structures are arranged in front of and behind the fragrance diffuser in an air flow direction, such that mounting stability of the fragrance diffuser in the air duct is ensured.

In an embodiment, the functional purifying member is arranged on a first side of the mounting frame, the fragrance diffuser is arranged on a second side of the mounting frame, the first side of the mounting frame and the second side of the mounting frame are arranged opposite each other; and/or in an air flow direction of the air duct, the fragrance diffuser is arranged on a side, facing an air outlet of the air duct, of the mounting frame.

It can be seen that in a case that the primary-efficiency filter screen and the fragrance diffuser are both arranged on the mounting frame, the primary-efficiency filter screen and the fragrance diffuser are arranged in the air duct through the same pluggable opening, and the number of pluggable openings provided on the casing is reduced, such that a layout of the casing is simpler. The fragrance diffuser is arranged closer to the air outlet of the air duct, such that the situation that since the fragrance diffuser excessively interferes with arrangement of components in the air duct, an air purifying effect in the air duct is affected is avoided.

In an embodiment, a first clamping block and a limiting plate are arranged on the mounting frame, a limiting step is arranged at the pluggable opening, and a second clamping block is arranged on an inner side wall of the air duct; when the mounting frame moves towards the pluggable opening and passes through the pluggable opening, coordinated abutting is performed after the first clamping block passes the second clamping block, and the limiting plate abuts against the limiting step; and when the mounting frame passes through the pluggable opening and moves away from the pluggable opening, the first clamping block passes the second clamping block and moves away from the second clamping block.

It can be seen that when the mounting frame passes through the pluggable opening and enters the air duct, the first clamping block is driven to pass the second clamping block. At the same time, the limiting plate abuts against the limiting step, such that the second clamping block is located between the first clamping block and the limiting plate. Moreover, the limiting plate abuts against the limiting step, such that the situation that the mounting frame falls into the air duct and cannot be taken out is avoided. Further, through mutual cooperation of the first clamping block and the second clamping block, in a working process of the purifier, the mounting frame is prevented from being ejected from the air duct. When the mounting frame is dismounted, the mounting frame is driven to pass through the pluggable opening and move away from the pluggable opening, a clamping structure between the first clamping block and the second clamping block is driven to be released, and the mounting frame is pulled out of the air duct.

In an embodiment, power of the second fan is higher than power of the first fan, and the fragrance diffuser is located in the first air duct.

It can be seen that two air ducts are arranged in the air purifier. The fan having the lower power is arranged in the first air duct, can be used for a long time, and is used for air purification. The fragrance diffuser is located in the first air duct. As the first fan in the first air duct works for a long time, the fragrance diffuser continuously releases a fragrance to a room, such that an indoor comfort level is improved. The fan in the second air duct has the higher power, and is used for hand drying.

In an embodiment, the first air duct and the second air duct each correspond to at least one pluggable opening.

It can be seen that the two air ducts both correspond to the pluggable openings, and functional purifying members are arranged in the air ducts respectively such that the air in the two air ducts can be purified.

In an embodiment, the casing includes an outer casing, a second inner casing and a first inner casing, where the air inlet, the second air outlet and the first air outlet are provided on the outer casing, and the second air duct and the first air duct are arranged in the outer casing; the second air duct penetrates the second inner casing; and the first air duct penetrates the first inner casing.

It can be seen that the two air ducts are formed in the outer casing, the two air ducts are completely independent from each other or partially overlap, and different setting modes and working modes are formulated according to an overall size of a hand dryer.

In an embodiment, the second fan and the first inner casing are arranged in a preset direction, the preset direction is perpendicular to an axial direction of the second fan; the axial direction of the second fan extends along a thickness direction of the outer casing; at least part of the first air duct and at least part of the second air duct are arranged in the thickness direction of the outer casing and parallel.

It can be seen that the second air duct and the first air duct are divided into two parts by the first inner casing in the second inner casing, such that a layout of components in the second inner casing is more compact, and a size of the hand dryer can be more effectively controlled. At least part of the second air duct is perpendicular to the axial direction of the second fan and the thickness direction of the outer casing. Moreover, at least part of the first air duct is perpendicular to the axial direction of the second fan and the thickness direction of the outer casing, such the space of the outer casing in the thickness direction are fully used by the two air ducts, so that the inside space of the outer casing is effectively utilized.

In addition, the second fan and the first inner casing are arranged in the preset direction, the preset direction is perpendicular to an axial direction of the second fan, such the space of the outer casing in the present direction are fully used by the second fan and the first inner casing, so that the inside space of the outer casing is effectively utilized.

That is to say, the thickness of the outer casing is basically the same as the axial height of the second fan, so that the space in the thickness direction of the outer casing are fully used by the second fan, so that the air purifier is not too thick, and there is no excess space in the thickness direction, which is easy achieved the miniaturization of the product. In addition, the second fan and the first inner casing are arranged in the direction perpendicular to the thickness direction of the outer casing. It also makes full use of the space in the outer casing, so that the components in the outer casing have the characteristics of compact structure.

In an embodiment, a first communication port is provided on the second inner casing, a first air duct segment is arranged on the second air duct and is located between the air inlet and the first communication port, the first air duct segment is arranged around a periphery of the second inner casing, a second air duct segment is arranged on the second air duct and is located between the first communication port and the second air outlet, and the second air duct segment penetrates the second inner casing; and/or a second communication port is provided on the first inner casing, a third air duct segment is arranged on the first air duct and is located between the air inlet and the second communication port, the third air duct segment is arranged around a periphery of the first inner casing, a fourth air duct segment is arranged on the first air duct and is located between the second communication port and the first air outlet, and the fourth air duct segment penetrates the first inner casing.

It can be seen that the air duct, the second inner casing and the first inner casing are arranged in the outer casing, and the second air duct is formed from the air inlet of the outer casing passing through the second inner casing to the second air outlet, the first air duct is formed from the air inlet of the outer casing passing through the first inner casing to the first air outlet. The second air duct and the first air duct naturally and correspondingly surround a periphery of the second inner casing or the first inner casing, such that an air intake area and an air intake volume are increased. In this way, the two air ducts pass through the first inner casing and the second inner casing respectively, and the flow paths of the two air channels are reasonably planned, and the space in the outer casing is fully utilized.

In an embodiment, two air inlets are provided on the outer casing, the second inner casing and the first inner casing are arranged between the two air inlets, and two first air duct segments are correspondingly formed between the outer casing and the second inner casing.

It can be seen that two first air duct segments and two third air duct segments are formed on the outer casing, such that an air duct area and an air intake volume are further increased.

In an embodiment, the air purifier further includes an air suction assembly and a functional purifying member, where the air suction assembly and the functional purifying member are arranged in the outer casing.

It can be seen that the air suction assembly is used for sucking the air outside the machine into the air duct, and the air is purified by the functional purifying member in the air duct, and then flows out of the machine through the air outlet. By means of the air duct assembly, a hand drying function and an air purifying function are independent from each other, such that hand drying efficiency is improved, and the hand dryer has the air purifying function. Moreover, wear and tear of the fans is reduced, and service life of the fans is prolonged.

In an embodiment, the functional purifying member includes a plasma assembly, and the plasma assembly is arranged in the second air duct or the first air duct; and/or the functional purifying member includes an anion assembly, the anion assembly is arranged in the second air duct or the first air duct, the anion assembly includes an anion generator and an anion high-voltage member, and the anion generator is connected to the anion high-voltage member; and/or the functional purifying member includes ultraviolet C (UVC), and the UVC is arranged in the second air duct or the first air duct.

It can be seen that the air in the air ducts can be sterilized and disinfected by the functional purifying members having different sterilization and disinfection principles.

In an embodiment, the plasma assembly includes a high-energy plasma tube and a plasma high-voltage pack, and the plasma high-voltage pack is connected to the high-energy plasma tube; and/or the plasma assembly includes a low-energy ion generator and a low-energy plasma high-voltage pack, and the low-energy ion generator is connected to the low-energy plasma high-voltage pack.

In an embodiment, the high-energy plasma tube or the low-energy ion generator is arranged in the first air duct; in an air flow direction of the first air duct, the first fan is arranged between the high-energy plasma tube or the low-energy ion generator and the first air outlet; and/or in a radial direction of the second fan, the first fan is arranged between the second fan and the first air outlet, and the radial direction of the second fan is perpendicular to an axis of a rotation center of the second fan; and/or the first fan is a centrifugal fan.

It can be seen that the first air outlet is provided closer to a fixed base, in contact with a wall, of the outer casing, such that the first air duct is suitable for an air purifying mode in which the air flows at a low speed, and the high-energy plasma tube or the low-energy ion generator is arranged in the first air duct. The fan in the first air duct is a centrifugal fan, such that even if the first air duct is bent, an air outlet volume can be continuously and effectively maintained, and an indoor air purification cycle is achieved.

In an embodiment, in a radial direction of the second fan, an air outlet of the second fan and the second air outlet are sequentially arranged, and the radial direction of the second fan is perpendicular to the axis of the rotation center of the second fan; and/or the second air outlet is provided farther away from a fixed base of the outer casing than the first air outlet; and/or the second fan is a series excitation fan.

It can be seen that the fixed base of the outer casing is in contact with the wall, the series excitation fan has high power and a large air volume, and the flowing air turns at an air outlet of the series excitation fan. The series excitation fan is arranged in the second air duct, such that the second air outlet has a greater air volume, and is used for a hand drying mode. Compared with the first air outlet, the second air outlet for the hand drying mode is farther away from the fixed base of the outer casing such that hands can be prevented from making contact with the wall. Moreover, due to the structure of the series excitation fan, the outer casing has more space for formation of two air ducts, such that a layout in the outer casing is more reasonable.

In an embodiment, a fan base is arranged in the outer casing, a through hole is provided on the fan base, the second fan is arranged on the fan base, and the through hole is in communication with a fan air inlet of the second fan; a ventilation cavity is provided between the fan base and a mounting surface, opposite the fan base, of the outer casing, and the second air duct sequentially passes through the ventilation cavity, the through hole, the second fan and the second air outlet; and/or in an air flow direction of the second air duct, a width of the second air duct closer to the second air outlet is gradually reduced; and the first air outlet includes a plurality of air outlet holes, and the plurality of air outlet holes are arranged in a circle.

It can be seen that the fan base is used for supporting the fan mounted in the air duct, such that when the air passes through the ventilation opening, enters the ventilation cavity, gathers and enters the second fan, the air in the air duct can more smoothly flow. Since the width of the second air duct closer to the second air outlet is gradually reduced, an air outlet nozzle is formed, air outlet pressure is increased, air outlet strength is greater, and hand drying time can be shortened. Moreover, since the first air outlet is formed by a plurality of air outlet holes, the air can relatively slowly flow out, and the first air outlet is suitable for the long-term air purifying function.

In an embodiment, a heater is arranged in the second air duct or the first air duct; and/or an infrared sensor is arranged on the casing, and the infrared sensor and the second air outlet are located on the same side of the casing.

It can be seen that the heater is arranged in the air duct such that a temperature of the air from the heater to the air outlet can be increased, and hand drying efficiency is improved. By means of the fragrance diffuser box, the air flowing out of the machine can have a fragrance, such that a comfort level of a toilet is improved. The infrared sensor is used for detecting hands, such that it is convenient to start the hand dryer. The air is filtered by the functional purifying member in the second air duct or the first air duct such that the air can be more thoroughly purified.

In the disclosure, the air purifier includes the casing, the air inlet, the first air outlet and the second air outlet, wherein the air inlet, the first air outlet and the second air outlet are provided on the casing, the first air duct of the air ducts is arranged between the air inlet and the first air outlet, the second air duct of the air ducts is arranged between the air inlet and the second air outlet, the first air duct and the second air duct are separately arranged in the casing, the first fan is arranged in the first air duct, and the second fan is arranged in the second air duct. The first air outlet and the second air outlet are arranged on the same side of the casing. The second fan includes the fan air inlet, the fan ventilation channel and the plurality of fan air outlets, the fan air inlet and the plurality of fan air outlets are in communication with the fan ventilation channel, the fan ventilation channel extends in the rotation axis of the second fan, the plurality of fan air outlets are located in the radial direction of the fan ventilation channel. A first direction and a second direction are parallel to the radial direction of the second fan. In the first direction, the fan air inlet and the first air outlet are sequentially arranged. In the second direction, the fan air outlet and the second air outlet are sequentially arranged. A air duct section are communication between the fan air outlet and the second air outlet in the second air duct, the air duct section and the first air duct are formed two layer in the extend direction of the axial direction of the second fan. The first fan is arranged between the second fan and the first air outlet. The radial direction of the second fan is perpendicular to an axis line of rotation of the second fan.

### BRIEF DESCRIPTION OF THE DRAWINGS

FIG. 1 is a solid diagram of an example of an air purifier according to an example of the disclosure.

FIG. 2 is an exploded view of the example of the air purifier in FIG. 1.

FIG. 3 is a structural diagram showing that a casing is separated from a bottom cover according to the example of the air purifier in FIG. 1.

FIG. 4 is a structural diagram showing that a casing is separated from an upper cover according to the example of the air purifier in FIG. 1.

FIG. 5 is a structural diagram showing that a top cover and a cover plate are opened according to the example of the air purifier in FIG. 1.

FIG. 6 is a sectional view of the example of the air purifier in FIG. 1.

FIG. 7 is a structural diagram showing that the bottom cover is removed according to the example of the air purifier in FIG. 1.

FIG. 8 is another sectional view of the example of the air purifier in FIG. 1.

FIG. 9 is a structural diagram showing that a low-energy plasma generator is mounted according to the example of the air purifier in FIG. 1.

FIG. 10 is a structural diagram showing that a top cover is removed according to the example of the air purifier in FIG. 1.

FIG. 11 is an exploded view of a mounting frame and a fragrance diffuser according to the example of the air purifier in FIG. 1.

FIG. 12 is an enlarged view of portion A in FIG. 7.

FIG. 13 is a solid diagram of an example of an air purifier according to another example of the disclosure.

FIG. 14 is a diagram of an air flow direction according to the example of the air purifier in FIG. 13.

FIG. 15 is an internal view of an inner casing according to the example of the air purifier in FIG. 13.

### REFERENCE NUMERALS

01. functional purifying member, 011. high-energy plasma tube, 012. ozone removal screen, 013. first fan, 014. primary-efficiency filter screen, 015. high-efficiency particulate air (HEPA) filter screen, 016. fragrance diffuser, 0160. limiting chamber, 0161. supporting plate, 0162. baffle, 01611. first opening, 01610, second opening, 0163. air hole, 017. low-energy plasma generator, 03. first air duct, and 04. second air duct;
1. bottom cover, 11. second seal groove, 111. first air duct segment, 121. third air duct segment, 112. second air duct segment, 122. fourth air duct segment, 113. air duct narrowing segment, 1131. connecting plate, 1132. ultraviolet lamp, 123. fragrance diffuser box, 13. printed circuit board (PCB) assembly, and 14. temperature sensor;
2. casing, 21. outer casing, 22. inner casing, 23. first seal groove, 24. second sealing plate, 231. fan cover, 212. fan ventilation channel, and 23. air suction assembly;
3. upper cover, 31. screw, 310. mounting frame, 3101. framework, 3102. limiting block, 3103. limiting concave portion, 3104. concave opening, 3105. opening, 311. second grabbing recess, 312. third grabbing recess, 32. second mounting opening, 33. cover plate, 331. first sealing member, 332. second sealing member, 34. partition block, 341. hollow portion, 35. recess, 36. first sealing plate, 37. air duct block, 371. heater, 38. pluggable opening, 380. first pluggable opening, 382. limiting step, 383. second pluggable opening, 381. limiting plate, 39. first grabbing recess, 3211. plasma assembly, 321. high-energy plasma tube, and 322. plasma high-voltage pack;
4. top cover, 400. outer casing, 42. fixed base, 41. outer casing structure, 43. mounting surface, 44. infrared sensor, and 42. fixed base;
5. mounting chamber, 51. first mounting chamber, 511. first side mounting plate, 512. second side mounting plate, 513. snap-fit groove, 514. screw hole, 515. electrically-conductive mounting position, 516. limiting convex rib, 517. limiting groove, 52. second mounting chamber, 521. second fan, 5211. fan air outlet, 5212. fan air inlet, 53. third mounting chamber, 500. second inner casing, 50. first communication port, 5201. fan guard, 5101. inner mounting base, 5311. fan base, 531. through hole, and 54. ventilation cavity;
6. air inlet, 61. first mounting opening, 62. first ventilation opening, 63. second ventilation opening, 64. third ventilation opening, 65. first air inlet, 66. second air inlet, 600. first inner casing, and 610. second communication port;
7. air outlet, 71. first air outlet, 72. second air outlet, and 73. air outlet;
8. electrically-conductive assembly, 81. movable electrically-conductive member, 811. step, 813. fixed block, 812. first limiting groove, 814. second limiting groove, 82. spring, 83. limiting block, and 84. limiting chamber; and
9. control assembly, and 91. air outlet hole.

The disclosure will be further described below in combination with accompanying drawings and examples.

### DETAILED DESCRIPTION OF THE EMBODIMENTS

A main objective of the disclosure is to provide an air purifier which can reduce wear and tear of fans.

In order to realize the above main objective, the disclosure provides an air purifier. The air purifier includes a casing 2, an air inlet 6, a first air outlet 71 and a second air outlet 72, where air ducts are arranged in the casing 2, the air inlet 6, the first air outlet 71 and the second air outlet 72 are provided on the casing 2, a first air duct 03 of the air ducts is arranged between the air inlet 6 and the first air outlet 71, a second air duct 04 of the air ducts is arranged between the air inlet 6 and the second air outlet 72, the first air duct 03 and the second air duct 04 are arranged in the casing 2, a first fan 013 is arranged in the first air duct 03, and a second fan 521 is arranged in the second air duct 04.

From the above solution, it can be seen that the double air ducts are arranged in the casing 2, the first fan 013 is arranged in the first air duct 03, and the second fan 521 is arranged in the second air duct 04. The first fan 013 and the second fan 521 have different air volumes, the fan having a greater air volume is mainly applied to perform a hand drying function, and the fan having a less air volume is applied to perform an air purifying function. The fan having a high-speed air supplying function and the fan having a low-speed air purifying function are independent from each other, such that it is not required to frequently switch modes of the fans, excessive wear and tear of the fans are avoided, and service life of the fans is prolonged.

Specifically, the second air duct 04 is used for a hand drying function, and the first air duct 03 is used for an air purifying function.

In addition, another objective of the disclosure is to rapidly add a new fragrance diffuser and replace a purifying assembly.

In order to realize the above main objective, the air purifier further includes at least one of a functional purifying member 01 and a fragrance diffuser 016, where at least one pluggable opening 38 is provided on the casing 2, and the functional purifying member 01 and/or the fragrance diffuser 016 passes through the at least one pluggable opening 38 so as to be mounted in the air duct by means of a limiting structure.

From the above solution, it can be seen that the functional purifying member 01 and the fragrance diffuser 016 are arranged in the air duct through the pluggable opening 38, such that the fragrance diffuser 016 and the functional purifying member 01 are mounted in the air duct in an open manner. The fragrance diffuser 016 and the functional purifying member 01 are mounted or dismounted in a pluggable manner such that the fragrance diffuser 016 and the functional purifying member 01 can be rapidly replaced.

On the basis of the objectives of the above two aspects, the disclosure provides two particular examples to achieve the above functions. In the following, the two solutions will be described in detail.

### Example 1

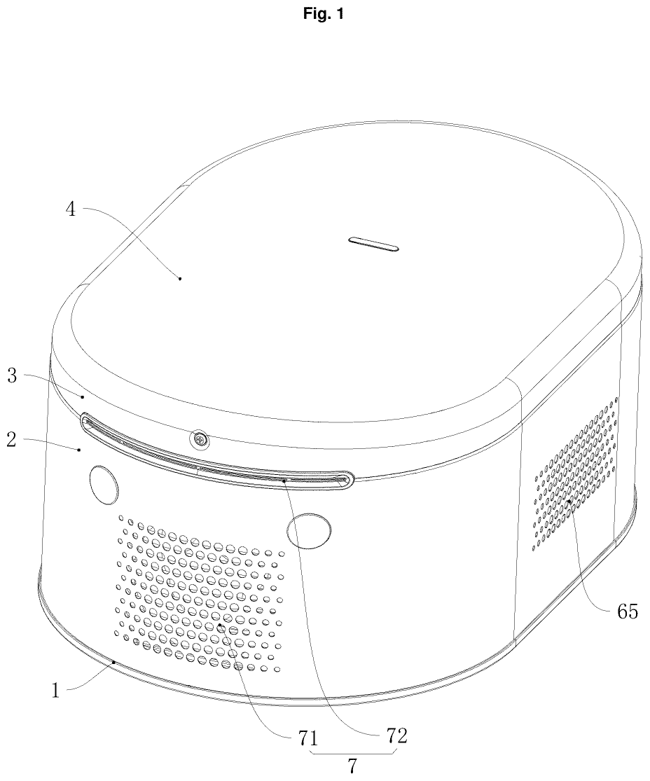

FIGS. 1-12 constitute the technical solution of Example 1.

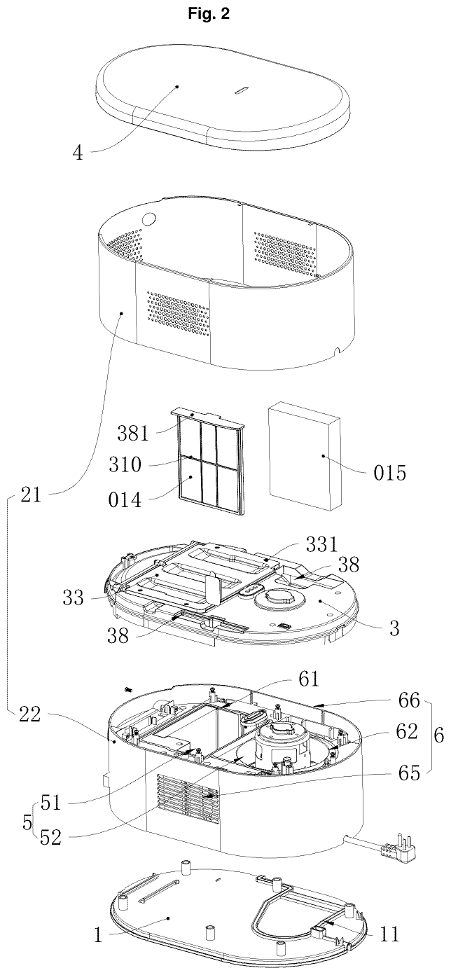

In particular examples shown in FIGS. 1 and 2, an air purifier includes a bottom cover 1, a casing 2, an upper cover 3 and a top cover 4. The casing 2 includes an outer casing 21 and an inner casing 22, and the inner casing 22 is located in the outer casing 21. The bottom cover 1, the casing 2, the upper cover 3 and the top cover 4 are sequentially stacked, and the bottom cover 1, the outer casing 21, the inner casing 22, the upper cover 3 and the top cover 4 have the same projection shape in a stacking direction and have matched areas, that is, the bottom cover 1, the casing 2, the upper cover 3 and the top cover 4 have the approximately same area. When stacking is carried out, the bottom cover 1, the casing 2, the upper cover 3 and the top cover 4 are distinctly layered. Through such shape setting and stacking manner, the bottom cover 1, the casing 2, the upper cover 3 and the top cover 4 can be rapidly positioned and assembled, and an assembly speed is increased.

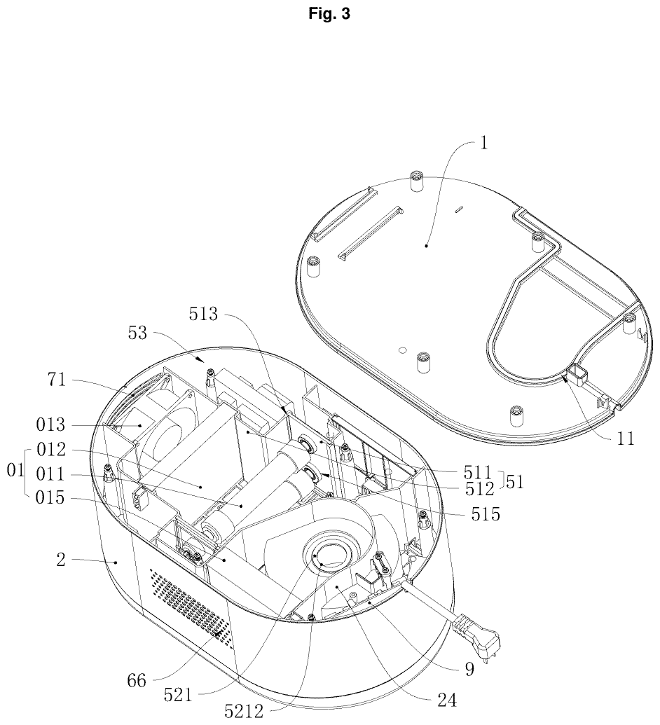

With reference to FIG. 3, at least one air inlet 6 and at least one air outlet 7 are provided on the casing 2, at least one mounting chamber 5 is arranged in the inner casing 22, and a fan and a functional purifying member 01 are arranged in the mounting chamber 5. The functional purifying member 01 is a component for purifying the air according to different purification principles, and the different purification principles may include filtration, sterilization, disinfection, interception, etc. An air duct is formed between the air inlet 6 and the air outlet 7 in a communication manner, and the air duct passes through the mounting chamber 5, such that at least one air duct is formed in the inner casing 22. Under the action of the fan in the mounting chamber 5, the air outside the air purifier enters the mounting chamber 5 through the air inlet 6, the functional purifying member 01 in the mounting chamber 5 sterilizes and disinfects the air entering the air purifier, so as to purify the air, and then the purified air is discharged through the air outlet 7.

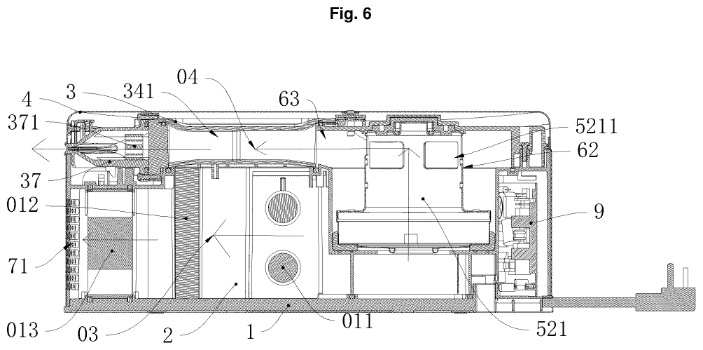

In the example, a first mounting chamber 51 and a second mounting chamber 52 are arranged in the inner casing 22, and different components are placed in the first mounting chamber 51 and the second mounting chamber 52. With reference to FIG. 6, in the example, a first air duct 03 and a second air duct 04 are formed in the inner casing 22, the first air duct 03 passes through the first mounting chamber 51, the second air duct 04 passes through the second mounting chamber 52, and the first mounting chamber 51 and the second mounting chamber 52 are in communication with different air inlets 6 and air outlets 7. The second air duct 04 is formed on a side, facing the casing 2, of the upper cover 3, such that the second air duct 04 is located between the first air duct 03 and the upper cover 3. In the example, a first air duct 03 and a second air duct 04 are formed in the inner casing 22, a first air inlet 65, a second air inlet 66 and a first air outlet 71 are formed on the inner casing 22, a second air outlet 72 is formed on the upper cover 3, the first air duct 03 is formed between the first air inlet 65 and the first air outlet 71, and the second air duct 04 is formed between the second air inlet 66 and the second air outlet 72. The first air inlet 65 and the second air inlet 66 are provided on two opposite sides of the inner casing 22 in a one-to-one correspondence manner, and the first air inlet 65 and the second air inlet 66 are provided opposite each other. The first air outlet 71 and the second air outlet 72 are located on the same side of the purifier. When the air purifier is arranged on a wall, the first air outlet 71 and the second air outlet 72 are located at a bottom of the purifier. On the basis of the bottom cover 1, the second air outlet 72 is located above the first air outlet 71, and the first air outlet 71 and the second air outlet 72 are located between the first air inlet 65 and the second air inlet 66. The first mounting chamber 51 is in communication with the first air inlet 65 and the first air outlet 71, and the second mounting chamber 52 is in communication with the second air inlet 66 and the second air outlet 72.

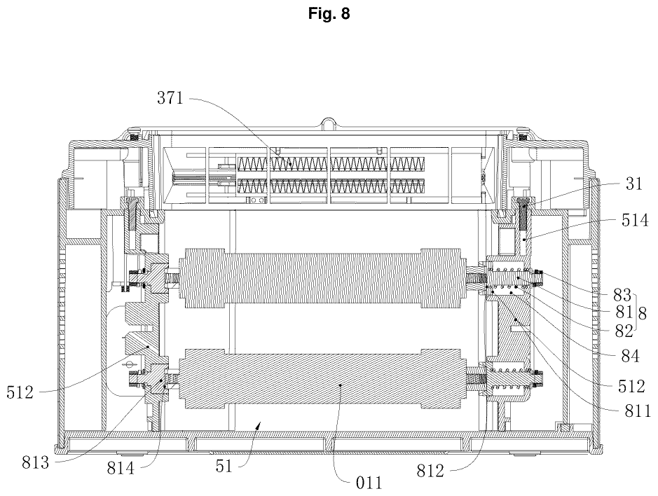

In the example, the first mounting chamber 51 is delimited by assembling and splicing a plurality of first side mounting plates 511 and two second side mounting plates 512, the plurality of first side mounting plates 511 are connected to each other in an integrally formed manner, and a side wall, facing the casing 2, of the bottom cover 1 is the bottom of the first mounting chamber 51. The first air duct 03 is located between the two second side mounting plates 512, the two second side mounting plates 512 are arranged opposite each other, the first side mounting plates 511 are fixed in the inner casing 22, and the second side mounting plates 512 are mounted in the casing 2 in a dismountable manner. The specific structure for achieving the dismountable second side mounting plates is as follows: snap-fit grooves 513 are provided on side walls, facing the second side mounting plates 512, of the first side mounting plates 511, and the second side mounting plates 512 are snap-fitted in the snap-fit grooves 513, such that the second side mounting plates 512 are arranged in the inner casing 22 in a dismountable manner. In order to ensure stability of the second side mounting plates 512 in a working process of the purifier, with reference to FIG. 8, screw holes 514 are provided on the second side mounting plates 512, screws 31 are arranged on the upper cover 3, and the screws 31 are connected to the screw holes 514 in a matching manner, such that the second side mounting plates 512 are connected to the upper cover 3. Due to the dismountable second side mounting plates 512, an area of the first mounting chamber 51 can be changed. The area of the first mounting chamber 51 can be adjusted such that an interior of the first mounting chamber 51 can adapt to mounting of functional purifying members 01 having different volumes and types. Another embodiment for changing the area of the first mounting chamber 51 is as follows: a side wall of the first mounting chamber 51 is delimited by the first side mounting plate 511, the first side mounting plate 511 is entirely fixed on the inner casing 22, and first side mounting plates 511 having different lengths delimit first mounting chambers 51 having different sizes, such that when different functional purifying members are replaced, the first side mounting plates 511 having different sizes are directly replaced in the inner casing 22. Therefore, first mounting chambers 51 having different areas can be obtained.

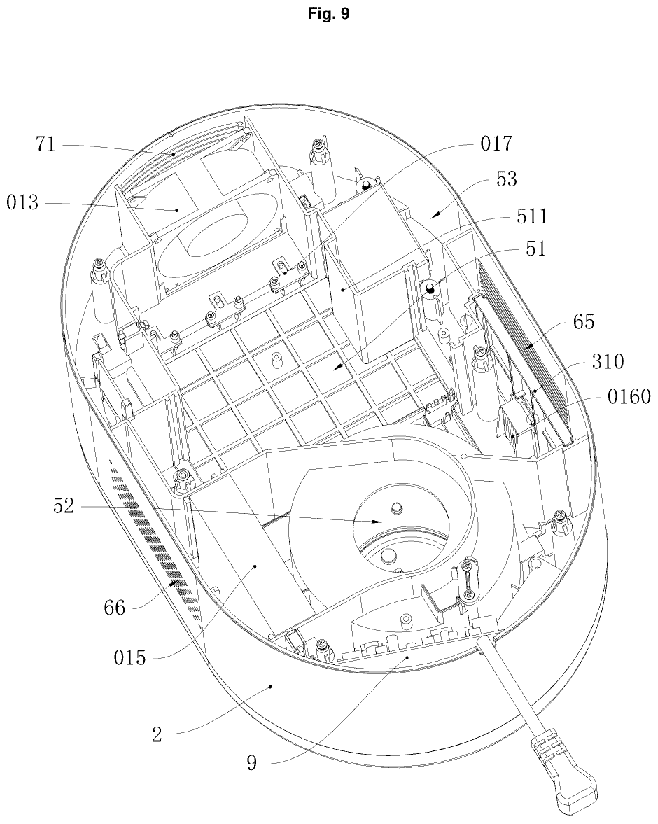

Due to the dismountable second side mounting plates 512, the area of the first mounting chamber 51 can be changed. In the example, a third mounting chamber 53 is further provided in the inner casing 22 through division, the third mounting chamber 53 is adjacent to the first mounting chamber 51, and the second side mounting plates 512 are located between the third mounting chamber 53 and the first mounting chamber 51. After the second side mounting plates 512 are dismounted, the first mounting chamber 51 is in communication with the third mounting chamber 53, such that the area of the first mounting chamber 51 is increased, and more space is provided for replacement of the functional purifying member 01. Specifically, a high-energy plasma tube 011 is originally arranged in the first mounting chamber 51, and a high-energy plasma mounting pack is arranged in the third mounting chamber 53. When it is required to replace the high-energy plasma tube 011 with a low-energy plasma generator, with reference to FIG. 9, one second side mounting plate is dismounted, such that the first mounting chamber 51 is in communication with the third mounting chamber 53, and then a low-energy plasma generator 017 and a mounting pack thereof are mounted in the first mounting chamber 51 having an increased area.

At least one electrically-conductive mounting position 515 is arranged on the side wall of the first mounting chamber 51, and the electrically-conductive mounting position 515 is arranged on the side wall of the first mounting chamber in various manners. For example, the electrically-conductive mounting position 515 is fixedly arranged on the first side mounting plate 511, or is arranged on the dismountable second side mounting plate 512. In a case that the electrically-conductive mounting position 515 is arranged on the second side mounting plate 512, the electrically-conductive mounting position 515 is arranged on at least one second side mounting plate 512 of the two second side mounting plates 512. Therefore, the high-energy plasma tube 011 or an ultraviolet lamp are kept electrified, so as to normally operate. In the example, the electrically-conductive mounting position 515 is arranged on the dismountable second side mounting plate 512. Since the electrically-conductive mounting position 515 is used for electrifying the functional purifying member, an electrified functional purifying member 01 is mounted in the first mounting chamber 51, and the electrified functional purifying member 01 includes a high-energy plasma tube 011. The low-energy plasma generator 017 or the ultraviolet lamp, the high-energy plasma tube 011 and the ultraviolet lamp are all tubular electrified functional purifying members. The electrically-conductive mounting position 515 is suitable for mounting of the high-energy plasma tube and the ultraviolet lamp such that the high-energy plasma tube 011 and the ultraviolet lamp can be alternately or both arranged on the at least one electrically-conductive mounting position 515. In the example, two electrically-conductive mounting positions 515 are arranged on the second side mounting plate 512. The two electrically-conductive mounting positions 515 are collinearly arranged in an extension direction from a top of the casing 2 to a bottom of the casing 2, such that space occupied by the electrified functional purifying member in the first mounting chamber 51 is reduced. High-energy plasma tubes 011 or ultraviolet lamps are arranged at two electrically-conductive mounting positions 515±, or a high-energy plasma tube 011 is arranged at one electrically-conductive mounting position, and an ultraviolet lamp is arranged at the other electrically-conductive mounting position.

With reference to FIG. 8, an electrically-conductive assembly 8 is arranged at the electrically-conductive mounting position 515, and the electrically-conductive assembly 8 includes a movable electrically-conductive member 81, a spring 82 and a limiting block 83. A limiting chamber 84 is arranged on the second side mounting plate 512, the movable electrically-conductive member 81 and the spring 82 are located in the limiting chamber 84, and the movable electrically-conductive member 81 is in clearance fit with the limiting chamber 84. The movable electrically-conductive member 81 penetrates the spring 82 and the second side mounting plate 512, a step 811 is arranged on the movable electrically-conductive member 81, and the spring 82 is arranged between the step 811 of the movable electrically-conductive member 81 and the second side mounting plate 512 in an abutting against manner. One end of the movable electrically-conductive member 81 is connected to a limiting block 83, and the limiting block 83 is located outside the first air duct 03. A first limiting groove 812 is provided on a side, facing the first air duct 03, of the movable electrically-conductive member 81. In the example, two opposite dismountable second side mounting plates 512 are arranged in the first mounting chamber 51, and electrically-conductive mounting positions 515 are arranged on the two second side mounting plates 512. An electrically-conductive assembly 8 is arranged at the electrically-conductive mounting position 515 of at least one second side mounting plate 512 of the two second side mounting plates 512, such that it is ensured that the high-energy plasma tube 011 or the ultraviolet lamp can be transversely mounted in the first mounting chamber 51. In the example, an electrically-conductive assembly 8 is arranged on one second side mounting plate 512, and a movable electrically-conductive member 81 is arranged on the second side mounting plate 512. No electrically-conductive assembly 8 is arranged at the electrically-conductive mounting position 515 of the other second side mounting plate 512, but a second limiting groove 814 is provided on the electrically-conductive mounting position by means of an immovable electrically-conductive fixed block 813. Specifically, an immovable fixed block 813 is arranged on the other second side mounting plate 512, and a second limiting groove 814 is provided on a side, facing the first limiting groove 812, of the fixed block 813. When it is required to mount the high-energy plasma tube 011 or the ultraviolet lamp in the first mounting chamber 51, a first axial end of the high-energy plasma tube 011 or the ultraviolet lamp is located in the first limiting groove 812, the movable electrically-conductive member 81 is driven to move, the spring 82 is pressed, and one end of the high-energy plasma tube 011 or the ultraviolet lamp is driven to move, such that a remaining part of the high-energy plasma tube 011 or the ultraviolet lamp completely enters the first mounting chamber 51. Moreover, a second axial end of the high-energy plasma tube 011 or the ultraviolet lamp is arranged in the second limiting groove 814, such that the high-energy plasma tube 011 or the ultraviolet lamp is transversely mounted in the first mounting chamber 51. In a process that the high-energy plasma tube 011 or the ultraviolet lamp is mounted, through cooperation between the movable first limiting groove 812 and the immovable second limiting groove 814, the high-energy plasma tube 011 or the ultraviolet lamp can be more rapidly balanced and stably mounted. On the electrically-conductive mounting position 515, by means of the matching structure of the movable electrically-conductive member 81 and the spring 82 of the electrically-conductive assembly 8, the high-energy plasma tube 011 or the ultraviolet lamp is mounted. The high-energy plasma tube 011 or the ultraviolet lamp is mounted without a mounting screw such that complexity can be avoided, and mounting is simpler and easier. By means of the spring 82, an electrically-conductive end of the high-energy plasma tube 011 can more tightly abut against the movable electrically-conductive member 81, and the electrically-conductive end of the high-energy plasma tube 011 can more easily conduct electricity.

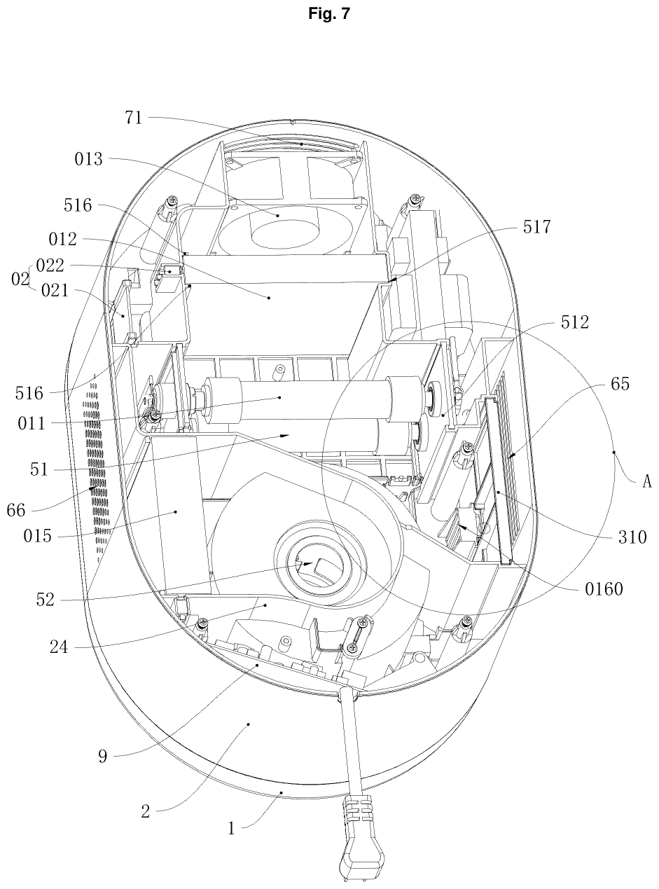

In a case that the functional purifying member is the high-energy plasma tube 011, since the high-energy plasma tube 011 is likely to generate ozone, if an ozone content is too high, quality of air purification is likely to be affected. An ozone removal screen 52 is arranged in the first mounting chamber 51, and the ozone removal screen 012 is arranged in the first mounting chamber 51 by means of a limiting structure. Specifically, with reference to FIG. 7, the limiting structure includes two limiting ribs 516, the two limiting convex ribs 516 are arranged on an inner side wall of the air duct, the two limiting convex ribs 516 are arranged opposite each other, and the ozone removal screen 012 is located between the two limiting convex ribs 516. On the basis of the inner side wall of the first mounting chamber 51, the two limiting convex ribs 516 should not have too great heights, such that flow of the air in the air duct is prevented from being excessively blocked. In order to improve stability of the ozone removal screen 012 in the first mounting chamber 51, a limiting groove 517 is provided on the other inner side wall of the first mounting chamber 51 in a concave manner, such that one side of the ozone removal screen 012 is located between the two limiting convex ribs 516, and the other side thereof is located in the limiting groove 517.

The fan used in the first air duct 03 is a first fan 013. In the example, the first fan 013 is arranged between the first air outlet 71 and the ozone removal screen 012. Specifically, the first fan 013 is an axial flow fan.

In the example, in an air flow direction of the first air duct 03, the air in the first air duct 03 enters the first mounting chamber 51 through the first air inlet 65, and sequentially passes through the electrified functional purifying member, the ozone removal screen 012, the first fan 013 and the first air outlet 71. In the air flow direction of the first air duct 03, a width of the first mounting chamber 51 is set in a small-great-small rule, that is, in the first mounting chamber 51, a width of the first air inlet 65 is less than a width of a position where the electrified functional purifying member and the ozone removal screen 012 are located, the width of the position where the electrified functional purifying member and the ozone removal screen 012 are located is greater than a width of a position where the first fan 013 is located, and the width of the position where the first fan 013 is located is substantially the same as a width of the first air outlet 71. Through such design structure, the air firstly passes through the position having the greater width in the first mounting chamber 51 where the electrified functional purifying member and the ozone removal screen 012 are arranged, such that the air gathers and is sterilized and disinfected by the functional purifying member. Then, under the action of the first fan 013, the purified air passes through the first fan 013 having the less width, such that a purification effect of the air discharged through the first air outlet 71 of the first air duct 03 is ensured.

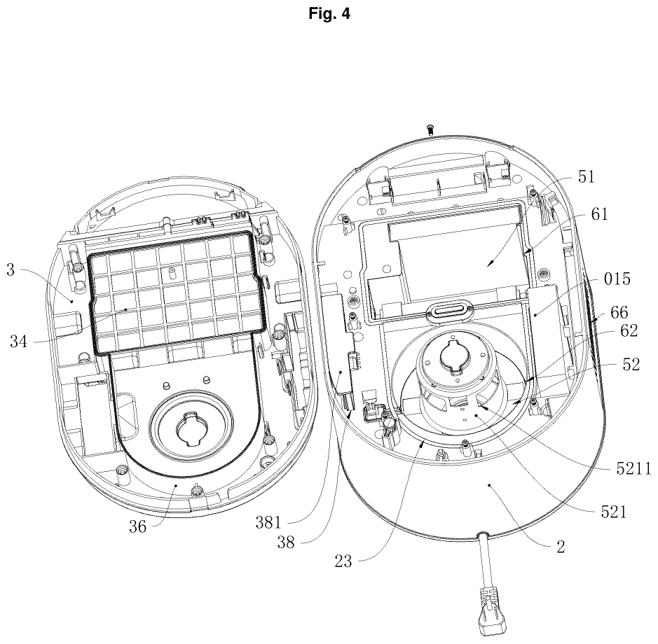

With reference to FIG. 4, a first mounting opening 61 and a first ventilation opening 62 are provided on a top surface, facing the upper cover 3, of the casing 2, the first mounting opening 61 is in communication with the first mounting chamber 51, and the functional purifying member 01 is arranged in the first mounting chamber 51. The functional purifying member 01 passes through the first mounting opening 61 and is arranged in the air duct, such that in a process of simple daily maintenance of the purifying hand dryer, components in the first mounting chamber 51 can be replaced through the first mounting opening 61 on a side wall, facing the upper cover 3, of the first mounting chamber 51. Moreover, it is not required to dismount the bottom cover 1 and the casing 2, such that a replacement speed is increased. The first ventilation opening 62 is in communication with the second mounting chamber 52, and a second fan 521 is arranged in the second mounting chamber 52. In the example, the second mounting chamber 52 and the second fan 521 match in shape. The second fan 521 is mounted in the second mounting chamber 52 in a manner as follows: the second mounting chamber 52 supports the second fan 521, sealing performance of the second fan 521 in the second mounting chamber 52 is ensured by a sealing member, and a soft shockproof rubber is arranged at a top, close to the upper cover 3, of the second fan 521, and is clamped between the second fan 521 and the upper cover 3. The functional purifying member 01 can be faster mounted in the air duct through the first mounting opening 61 in the first mounting chamber 51. In a case that double air ducts are formed on the casing 2, a second fan 521 in the second air duct 04 is arranged in the second mounting chamber 52, and the first ventilation opening 62 is one ventilation opening through which the second air duct 04 passes. Specifically, the second fan 521 is a series excitation fan.

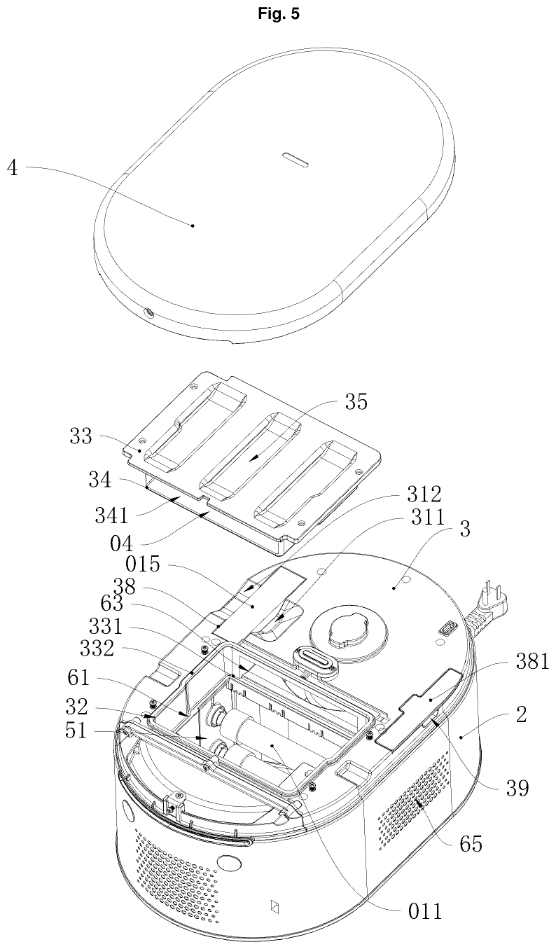

With reference to FIG. 5, a second mounting opening 32 is provided on the upper cover 3, the first mounting opening 61 is opposite and is in communication with the second mounting opening 32, and the cover plate 33 covers the second mounting opening 32. In a case that two air ducts are formed in the purifier, the first air duct is located in the casing, and the second air duct is located on a side, facing the casing 2, of the upper cover 3. A partition block 34 is arranged on the side wall, facing the casing 2, of the cover plate 33 in an outward protrusion manner, and on the basis of a surface, facing away from the top cover 4, of the cover plate 33, the partition block 34 has a certain thickness. A hollow portion 341 is arranged in the partition block 34, the hollow portion 341 penetrates the partition block 34 in an air flow direction of the second air duct 04, and the second air duct 04 is formed in the hollow portion 341. The partition block 34 covers the first mounting opening 61, and the second air duct 04 penetrates the partition block 34. Since the partition block 34 is arranged, and when the second air duct 04 is formed on the upper cover 3, the partition block 34 covers the first mounting opening 61, the first air duct 03 and the second air duct 04 are independent from each other. A first sealing member 331 is arranged between the cover plate 33 and the casing 2, and the first sealing member 331 is arranged in a circumferential direction of the first mounting opening 61. A second sealing member 332 is arranged between the cover plate 33 and the upper cover 3, and the second sealing member 332 is arranged in a circumferential direction of the second mounting opening 32. The sealing members further ensure sealing performance of the first air duct 03 and the second air duct 04. When the cover plate 33 covers the second mounting opening 32, the partition block 34 passes through the second mounting opening 32, and the partition block 34 covers the first mounting opening 61. Therefore, the partition block 34 abuts against the first sealing member 331, and sealing performance of the first air duct 03 is ensured. Moreover, the cover plate 33 abuts against the second sealing member 332 on the upper cover 3, such that sealing performance of the second air duct 04 is ensured. In addition, at least one grabbing recess 35 is arranged on a side wall, facing away from the first air duct, of the cover plate 33. By means of the recess 35, when the cover plate 33 is taken away from the casing 2, the cover plate 33 can be more easily picked up.

In the example, a first sealing plate 36 is arranged on a side, facing the casing 2, of the upper cover 3 in an outward protrusion manner, and the first sealing plate 36 is arranged in a circumferential direction of the first ventilation opening 62. A first seal groove 23 is provided on a side, facing the upper cover 3, of the casing 2, and the first sealing plate 36 is located in the first seal groove 23. On the basis of the side wall, facing the casing 2, of the upper cover 3, a projection height of the partition block 34 (that is, a thickness of the partition block 34 on the basis of a side, facing away from the top cover 4, of the cover plate 33) is the same as a projection height of the first sealing plate 36. A second ventilation opening 63 is provided between the first sealing plate 36 and the partition block 34, and the first ventilation opening 62 is in communication with the second ventilation opening 63. By means of the first sealing plate 36, the first ventilation opening 62 and the second ventilation opening 63 of the second mounting chamber 52 are raised, such that it is ensured that the second air duct can sequentially pass through the first ventilation opening 62, the second ventilation opening 63 and the partition block 34. Moreover, the partition block 34 and the first sealing plate 36 have the same height, such that sealing performance of the second air duct 04 is ensured. A second ventilation opening 63 is provided between the first sealing plate 36 and the partition block 34, and the first ventilation opening 62 is in communication with the second ventilation opening 63. A fan in the second air duct is the second fan 521, a fan air outlet 5211 of the second fan 521 is provided higher than a fan air inlet 5212 of the second fan 521, the second fan 521 is located in the second mounting chamber 52, and the fan air outlet 5211 of the second fan 521 passes through the first ventilation opening 62. The height setting structure of the fan air inlet 5212 and the fan air outlet 5211 of the second fan 521 better matches the upper-lower layered structure of the first air duct 03 and the second air duct 04. By means of the first sealing plate 36, the fan air outlet 5211 of the second fan 521 is provided opposite the first sealing plate 36, the fan air outlet 5211 of the second fan 521 is right opposite the second ventilation opening 63, and the air passes through the first ventilation opening 62 and flows into the partition block 34. An air duct block 37 is arranged on the upper cover 3, the second air duct 04 penetrates the air duct block 37, and the air duct block 37 is in communication with a second air outlet 72 of the second air duct of the upper cover 3. In an air flow direction of the second air duct 04, an air intake end of the air duct block 37 abuts against the partition block 34, the hollow portion 341 is in communication with an interior of the air duct block 37, and an air outlet end of the air duct block 37 abuts against the second air outlet 72. In the air flow direction of the second air duct 04, a width of the air duct block 37 is gradually reduced, such that with the change of pressure in the air duct block 37, a speed of the air is increased, and the air sprays out from the air outlet 73 of the second air duct. Moreover, a heater 371 is arranged in the air duct block 37, such that a temperature of the air in the air duct block 37 is increased, hands can be faster dried, and a comfort level is higher when the hands are dried in relatively cold weather. Therefore, in the air flow direction of the second air duct 04, the air in the second air duct 04 enters the second mounting chamber 52 through the second air outlet 72, then flows from the fan air inlet 5212 of the second fan 521 to the fan air outlet 5211 of the second fan 521 (in this case, the second air duct 04 passes through the first ventilation opening 62), and sequentially passes the second ventilation opening 63, the hollow portion 341 of the partition block 34, the air duct block 37 and the second air outlet 72.

A second seal groove 11 is provided on a side, facing the casing 2, of the bottom cover 1, a second sealing plate 24 is arranged on a side, facing the bottom cover 1, of the casing 2, the second sealing plate 24 is correspondingly arranged in the second seal groove 11, the second sealing plate 24 is arranged in a circumferential direction of the third ventilation opening 64 provided on the second mounting chamber 52 and facing the bottom cover 1, and the second sealing plate 24 extends to the second air inlet 66 of the second air duct. In summary, with reference to FIG. 6, the air flow direction of the first air duct 03 is as follows: the air passes through the first air inlet 65 on the casing 2, passes through the first mounting chamber 51, and flows out from the first air outlet 71 on the casing. The air flow direction of the second air duct 04 is as follows: the air passes through the second air inlet 66 on the casing 2, enters the second mounting chamber 52, sequentially passes through the first ventilation opening 62, the second ventilation opening 63, the partition block 34 and the air duct block 37, and then flows out from the second air outlet 72 on the upper cover 3. In the example, the first air inlet 65 and the second air inlet 66 are provided opposite each other. In a case that the air purifier is hung on a wall, the first air outlet 71 and the second air outlet 72 are located at a bottom of the air purifier, and the first air outlet 71 and the second air outlet 72 are located between the first air inlet 65 and the second air inlet 66.

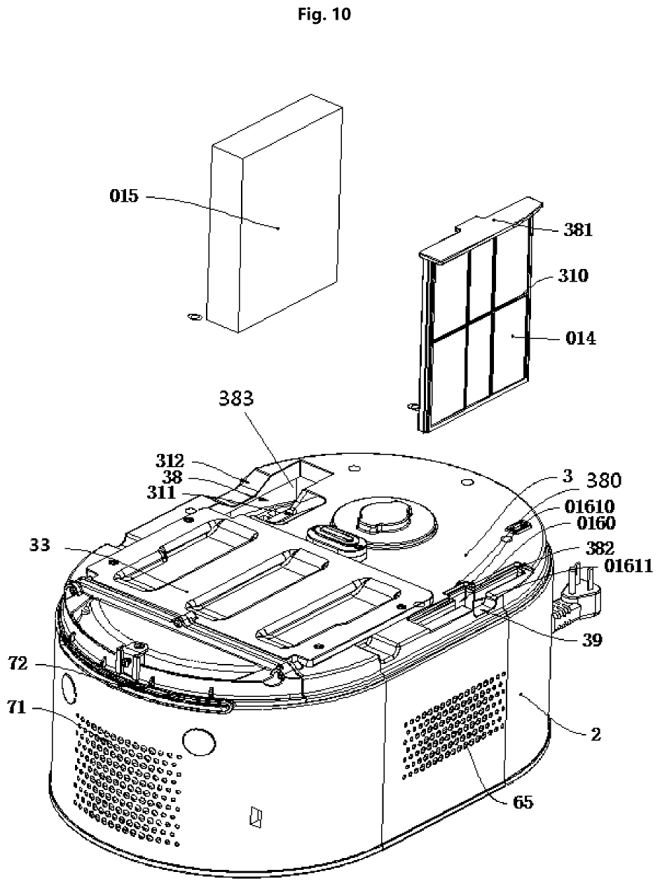

With reference to FIG. 10, at least one pluggable opening 38 is provided on the upper cover 3, the pluggable opening 38 extends into the inner casing 22, and the pluggable opening 38 is in communication with the first air duct 03 and the second air duct 04. The pluggable opening 38 and the second mounting opening 32 are located on the same side of the upper cover 3. In the example, the pluggable opening 38 and the second mounting opening 32 are provided on a side, facing the top cover 4, of the upper cover 3. The pluggable opening 38 and the first mounting opening 61 are provided on the same side of the upper cover 3, such that components in the air duct are arranged in the same direction, and a layout and mounting of the components in the air duct are simpler. In a case that the purifier does not include the upper cover 3, the pluggable opening 38 and the first mounting opening 36 are located on the same side, facing the top cover 4, of the casing 2. In the example, two pluggable openings are provided on the upper cover 3, and are a first pluggable opening 380 and a second pluggable opening 383 respectively, the first pluggable opening 380 is in communication with the first air duct 03, and the second pluggable opening 383 is in communication with the second air duct 04.

In the example, a non-electrified functional purifying member and/or a fragrance diffuser passes through the pluggable opening 38 so as to be mounted in the air duct by means of a limiting structure. The non-electrified functional purifying member is a primary-efficiency filter screen 014 and a high-efficiency particulate air (HEPA) filter screen 015.

The limiting structure includes a limiting plate 381, the limiting plate 381 is connected to the functional purifying member 01, the limiting plate 381 is located outside the first pluggable opening 380, and the limiting plate 381 is adapted to the pluggable opening 38. A first grabbing recess 39 is arranged on the casing 2, the first grabbing recess 39 is in communication with the first pluggable opening 380, and the limiting plate 381 is located above the first grabbing recess 39, such that the casing 2 supports the limiting plate 381. In this case, the functional purifying member 01 is a primary-efficiency filter screen 014, and the primary-efficiency filter screen 014 is arranged on a mounting frame 310. The mounting frame 310 includes a plurality of frameworks 3101, the plurality of frameworks 3101 are cross-connected to each other, the primary-efficiency filter screen 014 is fully unfolded, and the limiting plate 381 is connected to the mounting frame 310. In the example, the primary-efficiency filter screen 014 is located in the first air duct 03, and in the air flow direction of the first air duct 03, the primary-efficiency filter screen 014 is located between the first air inlet 65 and the electrified functional purifying member.

In a case that the functional purifying member is the HEPA filter screen 015, the functional purifying member 01 is in interference fit with the second pluggable opening 383. A second grabbing recess 311 and a third grabbing recess 312 are arranged on the casing 2, the second pluggable opening 383 is located in the second grabbing recess 311 and the third grabbing recess 312, and the second grabbing recess 311 and the third grabbing recess 312 are in communication with the second pluggable opening 383. In a case that the functional purifying member is arranged in an interference-fit manner, the functional purifying member can be better taken out through the second pluggable opening 383 by means of two grabbing recesses. In the example, the HEPA filter screen 015 is located in the second air duct 04, and the HEPA filter screen 015 is located between the second air inlet 66 and the second fan 521.

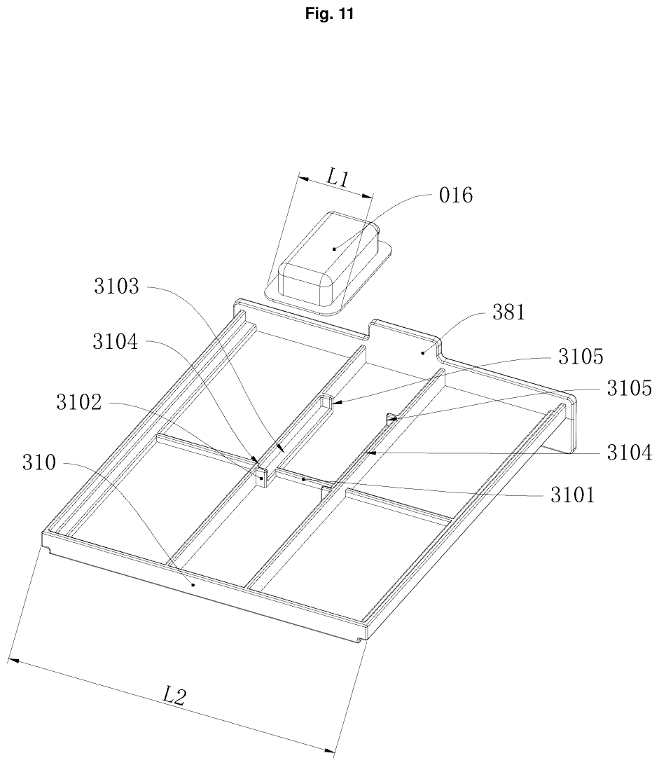

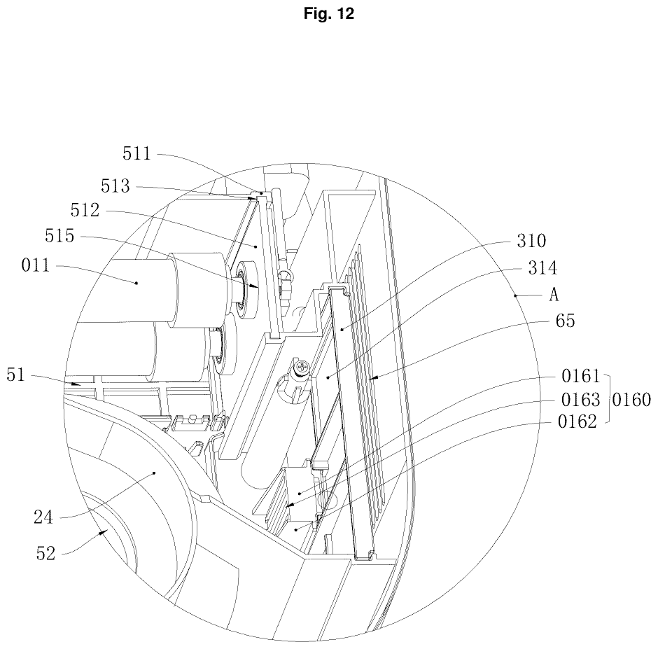

With reference to FIGS. 11 and 12, in a case that a fragrance diffuser 016 is arranged in the air duct through the pluggable opening 38, the limiting structure is a limiting chamber 0160, the fragrance diffuser 016 is arranged in the limiting chamber 0160, the limiting chamber 0160 is located in the first air duct 03, a first opening 01611 of the limiting chamber 0160 is in communication with the pluggable opening 38, a plurality of air holes 0163 are provided on a side wall of the limiting chamber 0160, and the air duct passes through the air holes 0163. In a case that the fragrance diffuser 016 is mounted in the air duct through the limiting chamber 0160, the fragrance diffuser sequentially passes through the pluggable opening 38 and the first opening 01611 of the limiting chamber 0160, and then enters the limiting chamber 0160. Since the air duct passes through the air holes 0163, when the air in the air duct flows, the air passes the fragrance diffuser 016 and passes, with a fragrance, through the air holes 0163, and then flows out of the device through the air outlet of the air duct. In the example, the fragrance diffuser 016 is located in the first air duct 03 for air purification.

The limiting chamber 0160 includes a supporting plate 0161 and a baffle 0162. The supporting plate 0161 serves as a bottom of the limiting chamber 0160, and the supporting plate 0161 is used for supporting the fragrance diffuser in the limiting chamber 0160. The baffle 0162 is connected to the supporting plate 0161, the baffle 0162 is arranged in a circumferential direction of the supporting plate 0161, such that the limiting chamber 0160 is delimited, and the baffle 0162 serves as a side wall of the limiting chamber 0160. The air holes 0163 are provided on a side wall, penetrating the baffle 0162, of the air duct, and the fragrance diffuser 016 is placed in the limiting chamber 0160, such that the fragrance diffuser 016 is mounted in the air duct.

The fragrance diffuser 016 may also be mounted in the air duct by means of a mounting frame 310. The mounting frame 310 includes a plurality of frameworks 3101, the plurality of frameworks 3101 are cross-connected to each other, the primary-efficiency filter screen 014 is arranged on the mounting frame 310, and the fragrance diffuser 016 is arranged on the mounting frame 310 by means of a limiting concave portion 3103. Specifically, limiting blocks 3102 are arranged on two adjacent and opposite frameworks 3101 respectively, one framework 3101 is connected between the two limiting blocks 3102, limiting concave portions 3103 are arranged on two limiting blocks 3102 respectively, concave openings 3104 of the two limiting concave portions 3103 have the same opening direction, and openings 3105 are further provided on sides, facing each other, of the two limiting blocks 3102. Therefore, after the fragrance diffuser 016 passes through the concave openings 3104 of the limiting concave portions 3103 and is arranged in the limiting concave portions 3103, the fragrance diffuser 016 is clamped between the two limiting blocks 3102 through the two openings 3105, and the fragrance diffuser 016 is in interference fit with the two limiting blocks 3102.

In the example, the fragrance diffuser 016 is fixed by means of the limiting chamber 0160 and the mounting frame 310. Specifically, a limiting chamber 0160 is arranged at the corresponding pluggable opening where the mounting frame 310 is mounted, width L1 of the limiting chamber 0160 is less than width L2 of the pluggable opening 38, a second opening 01610 is provided on the limiting chamber 0160, the air duct passes through the second opening 01610, and the fragrance diffuser 016 is located at the second opening 01610 and is clamped between the baffle 0162 of the limiting chamber 0160 and the mounting frame 310. Therefore, after the mounting frame 310 on which the fragrance diffuser 016 is fixed passes through the pluggable opening, the fragrance diffuser 016 is located in the limiting chamber 0160, the mounting frame 310 is outside the limiting chamber 0160, the fragrance diffuser 016 is clamped between the limiting chamber 0160 and the mounting frame 310, and a fragrance diffuser that is not used is in clearance fit with the side wall of the limiting chamber 0160 in the limiting chamber 0160. In the example, the primary-efficiency filter screen 014 is further arranged on a first side of the mounting frame 310, the fragrance diffuser 016 is arranged on a second side of the mounting frame 310, the first side of the mounting frame 310 and the second side of the mounting frame 310 are arranged opposite each other, and in an air flow direction of the air duct, the fragrance diffuser 016 is arranged close to an air outlet of the air duct. In a case that the primary-efficiency filter screen and the fragrance diffuser 016 are both arranged on the mounting frame 310, the primary-efficiency filter screen 014 and the fragrance diffuser 016 can be mounted in the air duct through the same pluggable opening 38, and the number of pluggable openings 38 provided on the casing 2 is reduced, such that a layout of the casing 2 is simpler. As another embodiment, the limiting chamber 0160 and the mounting frame 310 on which the primary-efficiency filter screen 014 is mounted is arranged in the air duct and correspond to different pluggable openings 38.

A first clamping block and a limiting plate 381 are arranged on the mounting frame 310, a limiting step 382 is arranged at the pluggable opening 38, a second clamping block is arranged on an inner side wall of the air duct, the mounting frame 310 passes through the pluggable opening 38, coordinated abutting is performed after the first clamping block passes the second clamping block, and the limiting plate 381 abuts against the limiting step 382. When the mounting frame 310 passes through the pluggable opening 38 and enters the air duct, the first blocking block is driven to pass the second clamping block, such that a clamping structure between the first clamping block and the second clamping block is achieved. Moreover, the limiting plate 381 just abuts against the limiting step 382, such that the situation that the mounting frame 310 falls into the air duct and cannot be taken out is avoided. In addition, under the mutual cooperation of the first clamping block and the second clamping block, in a working process of the purifier, the mounting frame 310 is prevented from being ejected from the air duct. When the mounting frame 310 passes through the pluggable opening and moves away from the pluggable opening 38, the first clamping block passes the second clamping block and moves away from the second clamping block, the clamping structure between the first clamping block and the second clamping block is driven to be released, and the mounting frame 310 is pulled out of the air duct.

In the example, two air ducts are arranged in the air purifier, and power of the second fan 521 arranged in the second air duct 04 is higher than power of the first fan 013 in the first air duct 03. The first fan 013 in the first air duct 03 can be used for a long time and therefore is used for air purification. The fragrance diffuser 016 is located in the first air duct 03, and as the first fan in the first air duct 03 works for a long time, the fragrance diffuser 016 continuously releases a fragrance to a room, such that an indoor comfort level is improved. The fan in the second air duct 04 has the higher power, and therefore is used for hand drying. Thus, the fans can be protected, and cost is saved. Pluggable openings 38 are correspondingly provided on the two air ducts respectively, such that functional purifying members are arranged in the air ducts respectively, and the air in the two air ducts can be purified. In the example, the non-electrified functional purifying member mounted in the first air duct 03 for purification by passing through the pluggable opening 38 is the primary-efficiency filter screen 014, and the non-electrified functional purifying member mounted in the second air duct 04 for hand drying by passing through the pluggable opening 38 is the HEPA filter screen 015.

In a case that an air duct is arranged in the air purifier, it is required to arrange no upper cover 3 on the air purifier, and the cover plate 33 and the pluggable opening 38 are provided on the casing. Electrified functional purifying members such as the high-energy plasma tube 011, the low-energy plasma generator and the ultraviolet lamp are mounted in the air duct through the mounting opening, and non-electrified functional purifying members such as the primary-efficiency filter screen and the filter screen are arranged in the air duct through the pluggable opening 38. Different functional purifying members are arranged in the air duct through different openings, and therefore are distinguished from each other, and safety can be further ensured. Moreover, the top cover 4 covers the upper cover 3 or the casing 2 by combining a buckle structure and a screw, and the buckle structure and the screw are arranged in front of and behind the top cover 4 respectively, such that front-rear limitation of the top cover 4 on the upper cover 3 is achieved, and fixation of the top cover 4 is ensured. The cover plate 33 is fastened to the casing 2 by means of a plurality of screws 31, but only one screw is used on the top cover 4, which is less than the number of screws 31 used on the cover plate 33. The buckle structure on the top cover 4 can be unfastened as long as the buckle structure is strongly lifted and one screw is unfastened, while it is required to unfasten the screws 31 one by one to unfasten the cover plate 33, such that an unfastening difficulty of the buckle structure is higher than an unfastening difficulty of the screws 31. After a user simply opens the top cover, components in the pluggable opening 38 is directly pulled out and replaced, such that technical requirements are relatively low. In a safe situation, the components in the pluggable opening 38 are simply replaced. However, the electrified functional purifying member is locked in the air duct by means of the cover plate 33 and the screw 31, and more complex steps are required for opening, such that opening is not easy, and safety is further ensured. The cover plate 33 is mounted by means of a plurality of screws 31 such that the cover plate 33 can be more tightly arranged on the upper cover 3, and tightness of the second air duct 04 is ensured.

The functional purifying member is a high-energy plasma tube 011 or an ultraviolet lamp. The double-air-duct air purifier includes a control assembly 9 and keys, the heater and the functional purifying member are connected to the keys, the keys are connected to the control assembly, and the keys are arranged on a side, facing away from the casing, of the upper cover. The high-energy plasma tube 011, the ultraviolet lamp and the heater 371 are high-power electrical appliances, and startup and shutdown of the above three components are controlled by means of the keys, such that power safety of the above three components is ensured.

The air flow direction of the first air duct 03 is as follows: the air passes through the first air inlet 65 on the casing 2, enters the first mounting chamber, sequentially passes through the primary-efficiency filter screen 014, the fragrance diffuser 06, the high-energy plasma tube 011, the ozone removal screen 012, and the first fan 013, and finally flows out from the first air outlet 71 on the casing 2. The air flow direction of the second air duct 04 is as follows: the air passes through the second air inlet 66 on the casing 2, enters the second mounting chamber 52, passes the second fan 521 in the second mounting chamber 52, passes through the first ventilation opening 62, is discharged through the fan air outlet 5211 of the second fan 521, passes through the second ventilation opening 63, the hollow portion in the partition block 34 and the air duct block 37, and then flows out from the second air outlet 72 on the upper cover 3.

When the air purifier is mounted, the bottom cover 1 is in contact with a wall, a mounting plate is arranged on the bottom cover 1, the mounting plate is mounted on the wall by means of a screw, and the mounting plate is connected to the bottom cover 1 by means of a buckle structure. In order to facilitate better hand drying of a user, two air outlets 7 are provided on a side, facing the ground, of the device, an infrared sensor is arranged beside the air outlets 7, the infrared sensor is connected to the control assembly, and the two air inlets 6 are provided on left sides and right sides of the air outlets 7 respectively. In a process that the air purifier is normally used on a wall, if it is required to replace the components in the device, it is required to open the top cover 4 facing a user, such that the functional purifying member at the pluggable opening 38 can be replaced. Then, the cover plate 33 is opened, such that components in the first mounting chamber 51 can be replaced through the first mounting opening 61. The components in the casing 2 can be replaced without taking down the whole device from the wall, such that a replacement process is simple and convenient.

### Example 2

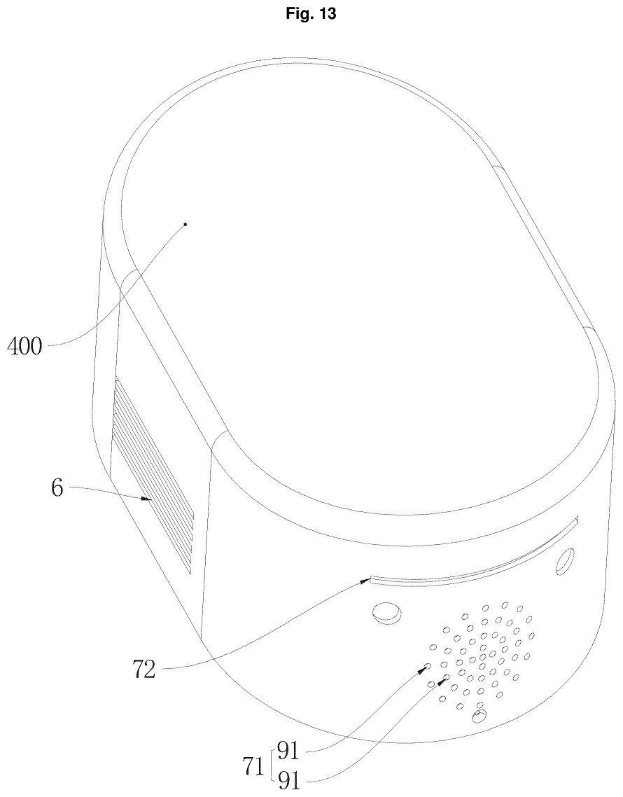

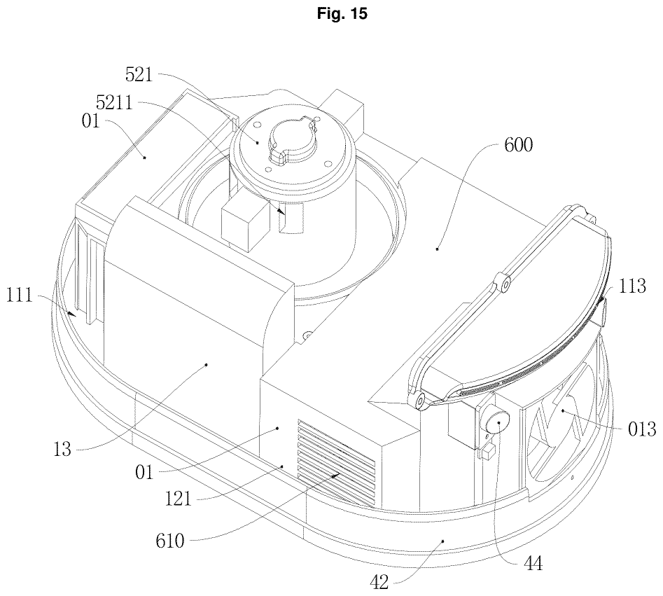

FIGS. 13-15 constitute the technical solution of Example 2. In particular examples shown in FIGS. 13-15, an air purifier having a hand-drying air duct and a purifying air duct is also provided.

The difference between Example 2 and Example 1 is that the double air ducts have different structures. Compared with Example 2, the technical solution of Example 1 has the feature that components in the double air ducts can be more conveniently mounted, replaced and disassembled.

From the perspective of appearance, with reference to FIGS. 13 and 1, the air purifier in Example 2 is designed to be in the same shape as the air purifier in Example 1, and both the shapes are elliptic cylinders. Therefore, processing is facilitated, and more beauty is achieved. When each of the air purifiers in the two examples is hung on a wall, air inlets are provided on two side walls of the air purifier respectively, two air outlets are located at a bottom of the device, and the air outlets are located between the air inlets on the two sides.

From the perspective of internal arrangement of the air purifier, the air suction assembly 200 of the casing includes an outer casing 400, a second inner casing 500, a first inner casing 600, an air inlet 6, a second air outlet 72 and a first air outlet 71, the second inner casing 500 and the first inner casing 600 are arranged in the outer casing 400, and the air inlet 6, the second air outlet 72 and the first air outlet 71 are provided on the outer casing 400. A second air duct 04 is arranged between the air inlet 6 and the second air outlet 72, and the second air duct 04 penetrates the second inner casing 500. A first air duct 03 is arranged between the air inlet 6 and the first air outlet 71, and the first air duct 03 penetrates the first inner casing 600. A second fan is arranged in the second air duct 04, and a first fan is arranged in the first air duct 03. The air suction assembly 200 and the functional purifying member 01 are arranged in the outer casing 400.

In the example, the outer casing 40 includes a fixed base 42 and an outer casing structure 41, and a plurality of mounting portions are arranged on the fixed base 42. The hand dryer is fixed on the wall by mounting screws on the plurality of mounting portions. The outer casing structure 41 is connected to the fixed base 42 along a periphery of the fixed base 42. The second inner casing 500 and the first inner casing 600 are fixed on the same inner side wall of the fixed base 42, and the inner side wall is a mounting surface 43 of the outer casing 400.

It can be seen therefrom that in the example, the second inner casing 500 and the first inner casing 600 that are independent from each other are arranged in the outer casing 400 in a spliced manner, cavities are provided in the second inner casing 500 and the first inner casing 600 respectively, and components are mounted and placed in the cavities. In an actual mounting process that the components are placed in the cavities and reversely buckled on the fixed base 42, mounting positions of the second inner casing 500 and the first inner casing 600 on the fixed base 42 are determined through mutual cooperation, and the second inner casing 500 and the first inner casing 600 are sequentially fixed on the fixed base 42 after the positions are determined. Therefore, the mounting process is complex.

Compared with Example 1, the outer casing 400 in the example is equivalent to a matching structure between the bottom cover 1, the casing 2 and the top cover 4 in Example 1. In Example 1, the upper cover 3 is added, and mounting manners of the inner casings 22 in the two examples are different. In order to more rapidly assemble the device, the inner casing 22 in Example 1 is a whole, the first mounting chambers 51 and the second mounting chambers 52 are provided at different positions on the inner casing 22 in Example 1, components are mounted in the first mounting chambers 51 and the second mounting chambers 52, and the second air duct 04 and the first air duct 03 pass through the different second mounting chambers 52 and first mounting chambers 51. However, the inner casing 22, the upper cover 3, the bottom cover 1, the top cover 4 and the outer casing match in shape, and are in shapes of elliptic cylinders respectively. In an actual mounting process, the bottom cover 1, the casing 2, the upper cover 3 and the top cover 4 are directly sequentially stacked, and are positioned through matching of the shapes. It is not required to repeatedly determine mounting positions of components to be spliced, and steps of fixing the components are reduced, such that an assembly speed is increased.

In the example, two air inlets 6 are provided on the outer casing 400, and the second inner casing 500 and the first inner casing 600 are arranged between the two air inlets 6. A first communication port 50 is provided on the second inner casing 500, and the second air duct 04 and the first communication port 50 form a first air duct segment 111 at the air inlet 6, such that two first air duct segments 111 are formed in the outer casing 400. Two second communication ports 610 are provided on the first inner casing 600, and the two second communication ports 610 are provided close to the two air inlets 6 in a one-to-one correspondence manner. In the example, the functional purifying member 01 includes a non-electrified functional purifying member, and the non-electrified functional purifying member is arranged in the second air duct 04 and/or the first air duct 03. A third air duct segment 121 is formed on the first air duct 03 and is located between one air inlet 6 and one second communication port 610, such that two third air duct segments 121 are formed in the outer casing 400. In the example, since on the basis of the mounting surface 43, a mounting height of the second inner casing 500 is close to an arrangement depth of the outer casing structure 41, and the two first air duct segments 111 are formed between the outer casing 400 and the second inner casing 500, the first air duct segment 111 naturally surrounds the periphery of the second inner casing 500 and is bent. Therefore, an air intake area and an air intake volume are increased. Since the second communication port 610 is provided closer to the air inlet 6 than the first communication port 50, a length of the third air duct segment 121 is less than a length of the first air duct segment 111.

A second air duct segment 112 is formed on the second air duct 04 and is located between the first communication port 50 and the second air outlet 72, and the second air duct segment 112 penetrates the second inner casing 500. A fourth air duct segment 122 is formed on the first air duct 03 and is located between the second communication port 610 and the first air outlet 71, and the fourth air duct segment 122 penetrates the first inner casing 600. The second air outlet 72 and the first air outlet 71 are provided between the two air inlets 6, and correspond, in a one-to-one manner, to the second air duct segment 112 and the fourth air duct segment 122 that are located between the two air inlets 6. In the example, the second inner casing 500 includes a fan guard 5201 and an inner mounting base 5101, the fan guard 5201 and the inner mounting base 5101 are connected to form the second air duct segment 112, and the fan guard 5201 and the inner mounting base 5101 are connected in an integrally formed manner or connected by means of a connecting member such as a screw. The inner casing 600 is a whole, and after the whole is deeply dug to form the fourth air duct segment 122 and the second communication port 610, the components are mounted and then reversely buckled on the fixed base 42. Therefore, the fourth air duct segment 122 is only in communication with the second communication port 610 and the first air outlet 71, and sealing performance is desirable.

From the perspective of the design structure of the double air ducts, the double air ducts in each of the example and Example 1 are independent from each other, and correspond to different air inlets and air outlets. Similarly, one air duct corresponds to an air-purifying air duct having a relatively low flow rate, and the other air duct corresponds to a hand-drying air duct having a relatively high flow rate. In each of the example and Example 1, relative positions of two air inlets and two air outlets are the same, and relative positions of two air ducts are the same; by means of the series excitation fan, the hand-drying air duct is turned; and on the basis of a mounting wall, the hand-drying air duct is located above the purifying air duct. However, the specific structure of the double air ducts in the example is different from the specific structure of the double air ducts in Example 1.

In the example, a second air duct 04 is arranged in the second inner casing 500, and a first air duct 03 is arranged in the first inner casing 600. The second air duct 04 is formed in a manner as follows: a first air duct segment 111 is formed between one air inlet 6 and the first communication port 50 on the second inner casing 500; the first communication port 80 of the second inner casing 500 and the second air outlet 72 form a second air duct segment 112; the first air duct segment 111 and the second air duct segment 112 are in communication with each other to form the second air duct 04; the second air duct 04 is connected to the fan guard 5201 by means of the inner mounting base 5101, such that sealing performance of the second air duct 04 in the second inner casing 500 is ensured; and an air duct narrowing segment 113 is formed by two connecting plates 1131 and is connected between the second inner casing 500 and the second air outlet 72. In addition, the first air duct 03 is formed in a manner specifically as follows: a third air duct segment 121 is formed between one air inlet 6 and the second communication port 610 on the first inner casing 600; a fourth air duct segment 122 is formed between the second communication port 610 and the first air outlet 71; the fourth air duct segment 122 is located in the first inner casing 600; the third air duct segment 121 and the fourth air duct segment 122 are in communication with each other to form a first air duct 03; and an air duct is formed by forming a cavity in the first inner casing 600. However, in the example, the second inner casing 500 is not integrally formed, and the air duct in the second inner casing 500 is formed by splicing the inner mounting base 5101, the fan guard 5201 and the connecting plate 1131, such that a splicing process is complex.

In Example 1, two first mounting chambers 51 and a second mounting chamber 52 are formed on the inner casing 22, and one air duct correspondingly passes through one mounting chamber. When the inner casing 22 matches the upper cover 3 and the top cover 4, the air duct can be formed. It is not required to splice various components having small sizes, such that assembly efficiency of the device is improved.

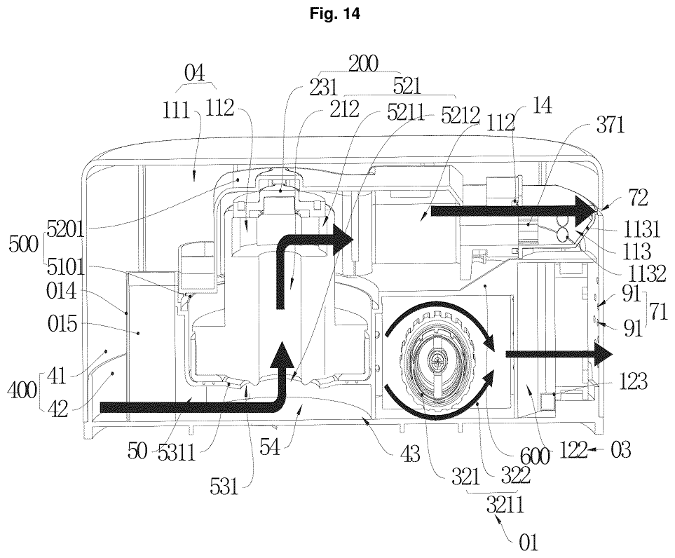

Moreover, with reference to FIGS. 14 and 6, a positional relationship between the first air duct and the second air duct, a position of the air inlet, a position of the air outlet, etc. in the double-air duct structure in the example are basically the same as those in Example 1, but specific arrangement structures of the first air duct and the second air duct are different. In the example, the components are mounted on the second inner casing 500 and the first inner casing 600, and the second inner casing 500 and the first inner casing 600 are arranged on the fixed base 42, and closed air ducts are formed in the second inner casing 500 and the first inner casing 600 such that the components in the air ducts can be dismounted only after the second inner casing 500 and the first inner casing 600 are dismounted. Therefore, operations are complex.

In Example 1, a plurality of openings (the pluggable opening 38 and the first mounting opening 61) are provided at the upper cover 3, and the openings are in communication with the air duct, such that the components in the air duct can be more conveniently mounted or dismounted. In Example 1, the first air duct 03 and the second air duct 04 that are independent from each other and sealed are formed by combining the mounting chamber on the inner casing and the partition block 34 with a hollow portion having a certain thickness.

In the example, the functional purifying member 01 includes a non-electrified functional purifying member, and the non-electrified functional purifying member is arranged in the second air duct 04 and/or the first air duct 03. The non-electrified functional purifying member includes a primary-efficiency filter screen 014 and a HEPA filter screen 015. In the air flow direction of the second air duct 04, the HEPA filter screen 015 is arranged farther away from the air inlet 6 than the primary-efficiency filter screen 014. In the example, the non-electrified functional purifying member in the second air duct 04 is arranged at the first communication port 50. In the air flow direction of the second air duct 04, the air enters the first air duct segment 111 through the air inlet 6, passes the non-electrified functional purifying member, and enters the second air duct segment 112 through the first communication port 50. An area of each of the primary-efficiency filter screen 014 and the HEPA filter screen 015 is greater than an area of the first communication port 50. The non-electrified functional purifying member in the first air duct 03 is arranged at the second communication port 610. In the air flow direction of the first air duct 03, the air enters the third air duct segment 121 through the air inlet 6, passes the non-electrified functional purifying member at the second communication port 610, and enters the fourth air duct segment 122.

A fan base 5311 is arranged in the second inner casing 500, and a through hole 531 is provided on the fan base 5311. The fan in the air suction assembly 200 is mounted in the fan base 5311, and moreover, the fan base achieves an air guiding function, such that the air entering the second inner casing 500 is guided to enter the air suction assembly 200. In the example, a ventilation cavity 54 is provided between the fan base 5311 and the mounting surface 43 of the outer casing 400, and the first communication port 50 is in communication with the ventilation cavity 54. In the air flow direction of the second air duct segment 112, the air sequentially passes through the ventilation opening, the ventilation cavity 54, the through hole 531 on the fan base 5311 and the air suction assembly 200.

The air suction assembly 200 includes a second fan 521 and a first fan 013, the second fan 521 is arranged in the second air duct 04, and the first fan 013 is arranged in the first air duct 03. In the example, the second fan 521 is a series excitation fan, and the first fan 013 is a centrifugal fan. An air outlet volume of the second fan 521 is greater than an air outlet volume of the first fan 013, such that the second fan 521 is mainly used for performing the hand drying function, and the first fan 013 is used for performing the air purifying function. The air suction assembly 200 includes a fan cover 231. The second fan 521 includes a fan air inlet 5212, a fan ventilation channel 212 and a plurality of fan air outlets 5211, the fan air inlet 5212 and the plurality of fan air outlets 5211 are in communication with the fan ventilation channel 212, the fan ventilation channel 212 extends in a rotation axis of the second fan 521, the fan air inlet 5212 is located at a first axial end of the fan ventilation channel 212, and the fan cover 231 is arranged at a second axial end of the fan ventilation channel 212. The plurality of fan air outlets 5211 are located in a radial direction of the fan ventilation channel 212, the fan air outlets 5211 are located between the fan air inlet 5212 and the fan cover 231, and the second air duct 04 sequentially passes through the fan air inlet 5212, the fan ventilation channel 212 and the plurality of fan air outlets 5211. The second fan 521 is arranged on the fan base 5311, and the through hole 531 is in communication with the fan ventilation channel 212. The second air duct segment 112 sequentially passes through the ventilation cavity 54, the through hole 531, the fan air inlet 5212, the fan ventilation channel 212, the plurality of fan air outlets 5211 and the second air outlet 72.

In the example, in a first direction, the fan air inlet 5212, the first inner casing 600 and the first air outlet 71 are sequentially arranged, and the first fan 013 is located in the first inner casing 600, such that the first fan 013 is arranged between the second fan 521 and the first air outlet 71. In a second direction, the fan air outlet 5211 and the second air outlet 72 are sequentially arranged. The first direction and the second direction are perpendicular to an axis of a rotation center of the second fan 521, and the first direction and the second direction are parallel to the radial direction of the second fan 521. In cooperation with the structure of the second fan 521, the second inner casing 500 is divided into two layers, and different components are arranged in each layer. The second air duct segment 112 forms at least two bent air ducts, and the first inner casing 600 is arranged at a corresponding bent position such that a space in the outer casing 400 can be fully used. Further, there is a distance between the second air outlet 72 and the first air outlet 71 on the basis of the mounting surface 43, and the second air outlet 72 and the first air outlet 71 have different arrangement heights on the basis of the mounting surface 43. In the example, the second air outlet 72 is located further away from the mounting surface 43 of the fixed base 42 of the outer casing 400 than the first air outlet 71. The fixed base 42 of the outer casing 400 is in contact with the wall, and the series excitation fan has high power and a large air volume, and the flowing air turns at the air outlet. The series excitation fan is arranged in the second air duct 04, such that the second air outlet 72 farther away from the fixed base 42 of the outer casing 400 than the first air outlet 71 has a greater air volume, is used for the hand drying mode, and prevents the hands from making contact with the wall. Due to the structure of the series excitation fan, there is more space in the outer casing 400 for formation of two independent and parallel air ducts, such that a layout in the outer casing 400 is more reasonable. The first air outlet 71 is provided closer to the fixed base 42 of the outer casing 400 in contact with the wall, such that the first air duct 03 is suitable for the air purifying mode in which the air flows at a low speed, and the first fan 013 in the first air duct 03 is a centrifugal fan. In the first direction, the first fan 013 is arranged between the second fan 521 and the first air outlet 71, and the first air duct 03 is bent, such that the air volume can be continuously and effectively maintained, and an indoor air purification cycle is achieved.

The functional purifying member 01 includes a plasma assembly 3211, and the plasma assembly 3211 is arranged in the second air duct 04 or the first air duct 03. In the example, the plasma assembly 3211 is arranged in the fourth air duct segment 122 of the first air duct 03. The plasma assembly 3211 includes a high-energy plasma tube 321 and a plasma high-voltage pack 322, and the plasma high-voltage pack 322 is connected to the high-energy plasma tube 321. In the air flow direction of the first air duct 03, the first fan 013 is arranged between the plasma assembly 3211 and the first air outlet 71. As another embodiment, the functional purifying member may further include at least one of ultraviolet C (UVC), an anion assembly, a bipolar ion generator or a low-energy ion generator. The anion assembly includes an anion generator and an anion high-voltage member, and the anion generator is connected to the anion high-voltage member. The low-energy ion generator is connected to the low-energy plasma high-voltage pack.

In the example, in the air flow direction of the second air duct segment 112, an air duct narrowing segment 113 is arranged at a position, close to the second air outlet 72, of the second air duct segment 112, and the air duct narrowing segment 113 is in communication with the second air duct segment 112 and the second air outlet 72. The air duct narrowing segment 113 is formed between two connecting plates 1131. One connecting plate 1131 is obliquely arranged, such that a ventilation width of the air duct narrowing segment 113 is gradually reduced, an air outlet nozzle is formed, air outlet pressure is increased, air outlet strength is greater, and hand drying time can be shortened. In order to further improve a degree of air purification, an ultraviolet lamp 1132 is arranged in the air duct narrowing segment 113.

Since the second fan 521 having a greater air volume is arranged in the second air duct 04, in order to adapt to the air duct narrowing segment 113 in the second air duct segment 112, the second air outlet 72 is elongated, and a width of the second air outlet 72 is substantially the same as a width of the air duct narrowing segment 113. The first fan 013 having a less air volume is arranged in the first air duct 03, the plasma assembly 3211 is arranged in the first air duct 03, and the first fan 013 may operate at a low speed for a long time. The first air outlet 71 includes a plurality of air outlet holes 91, the plurality of air outlet holes 91 are arranged in a circle or in a radial shape such that the air can slowly flow out, and the first fan is suitable for performing a long-term air purifying function.

In the example, a positive temperature coefficient (PTC) ceramic heater 371 is arranged in the second air duct 04, and can be used for increasing a temperature of the air from the PTC ceramic heater 371 to the second air outlet 72, such that hand drying efficiency is improved. A fragrance diffuser box 123 is arranged in at least one of the first air duct 03 and the second air duct 04. By means of the fragrance diffuser box, the air flowing out of the machine can have fragrance, and a comfort level of a toilet is improved. An infrared sensor 44 is arranged in the outer casing 400, a through hole is provided on the outer casing 400, and the infrared sensor 44 is arranged corresponding to the through hole. In the example, the infrared sensor 44, the second air outlet 72 and the first air outlet 71 are located on the same side of the outer casing 400, and the infrared sensor 44 is arranged between the second air outlet 72 and the first air outlet 71. The infrared sensor 44 is used for detecting hands such that the hand dryer can be started.

The air-purifying hand dryer includes a printed circuit board (PCB) assembly 13 and a temperature sensor 14, the PCB assembly 13 is arranged in the outer casing 400, and the PCB assembly 13 is electrically connected to the second fan 521, the first fan 013, the plasma high-voltage pack 322, the ultraviolet lamp 1132, the temperature sensor 14 and the infrared sensor 44. The temperature sensor 14 is arranged in the second air duct segment 112, and the temperature sensor 14 is arranged between the PTC ceramic heater and the second fan 521.

According to the air-purifying hand dryer, by arranging double ducts, the air can be purified no matter whether the hand dryer is in a hand-drying working state. The fans are arranged in the double ducts respectively, such that wear and tear of the fans is reduced, and service life of the fans is prolonged. The second fan 521 having a large air volume is arranged in the second air duct 04, and is mainly used for performing a hand drying function. A working flow of the second air duct 04 is as follows: when the infrared sensor 44 of the hand dryer senses human hands, the second fan 521 starts a maximum air volume mode, the air enters through the air inlet 6, passes through a non-electrified functional purifying member at the first communication port 50, and then sequentially passes the first communication port 50, the ventilation cavity 54, the through hole 531 of the fan base 5311 and the second fan 521. After passing through the high-speed fan, the air is accelerated. The air is heated by the PTC ceramic heater 371, and blows from the second air outlet 72 at a high speed by means of the air duct narrowing segment 113. A first fan 013 having a small air volume is arranged in the first air duct 03, and is mainly used for performing an air purifying function. A working flow in the first air duct 03 is as follows: the first fan 53 starts a low air volume and low noise mode, and the air enters the first inner casing 600 through the air inlet 6, and is sucked into a zone where the high-energy plasma tube 321 is located. In this high-concentration ion zone, the air is disinfected and sterilized. After passing through the low-speed first fan 013, the air blows from the first air outlet 71. Through circulating disinfection of the air, the whole space is disinfected and deodorized. In the hand drying mode, an air temperature can be measured by the temperature sensor 14. In a case that the air temperature is less than 25° C., the PTC ceramic heater 371 is started to provide warm air, such that a comfort level is improved. In a case that the air temperature is greater than 25° C., the PTC ceramic heater 371 is closed, such that energy is saved.

The differences between an alternative embodiment and the above embodiment are as follows: no purifying inner casing is arranged in the outer casing, and the hand-drying air duct and the purifying air duct penetrate the hand-drying inner casing. A ventilation opening is provided on the hand-drying inner casing, and the air duct segment formed between the hand-drying air duct and the ventilation opening and the air duct segment formed between the purifying air duct and the ventilation opening completely overlap. The hand-drying air duct and the purifying air duct are completely independent from each other in the hand-drying inner casing, or the hand-drying air duct and the purifying air duct may both pass through a fan ventilation channel in a second fan, such that the hand-drying air duct and the purifying air duct overlap in the fan ventilation channel, and then are divided into two parts at an air outlet and extend towards the second air outlet or the first air outlet respectively.

From the perspective of the interior of the device, in each of the example and Example 1, an electrified functional purifying member and a non-electrified functional member can be mounted, the non-electrified functional purifying member is at least arranged in the air duct for hand drying, and the electrified functional purifying member is correspondingly arranged in the air duct for air purification and having a slow flow rate. The difference is that the functional purifying member in the example is mounted on the fixed base in a closed manner, while the functional purifying member in Example 1 is mounted in the inner casing in an open manner by means of the pluggable opening 38, the first mounting opening 61 and the partition block 34.

Finally, it needs to be emphasized that the above examples are merely preferred examples of the disclosure, and are not intended to limit the disclosure. For those skilled in the art, various changes and modifications can be made on the disclosure, and any modifications, equivalent replacements, improvements, etc. made within the spirit and principles of the disclosure should fall within the scope of protection of the disclosure.

1. An air purifier, comprising a casing, an air inlet, a first air outlet and a second air outlet, wherein air ducts are arranged in the casing, the air inlet, the first air outlet and the second air outlet are provided on the casing, a first air duct of the air ducts is arranged between the air inlet and the first air outlet, a second air duct of the air ducts is arranged between the air inlet and the second air outlet, the first air duct and the second air duct are arranged in the casing, a first fan is arranged in the first air duct, and a second fan is arranged in the second air duct.

2. The air purifier according to claim 1, further comprising at least one of a functional purifying member and a fragrance diffuser, wherein at least one pluggable opening is provided on the casing, and the functional purifying member and/or the fragrance diffuser passes through the at least one pluggable opening so as to be mounted in the air duct by means of a limiting structure.

3. The air purifier according to claim 2, wherein the functional purifying member comprises at least one of a primary-efficiency filter screen, a high-efficiency particulate air (HEPA) filter screen and an ozone removal screen.

4. The air purifier according to claim 2, wherein
the limiting structure comprises a limiting plate, the limiting plate is connected to the functional purifying member, the limiting plate is located outside the pluggable opening, and the casing supports the limiting plate; and/or
the functional purifying member is in interference fit with the pluggable opening; and/or
the limiting structure comprises at least two limiting convex ribs, the at least two limiting convex ribs are arranged on an inner side wall of the air duct, the at least two limiting convex ribs are arranged opposite each other, and the functional purifying member is located between the at least two limiting convex ribs; and/or
the limiting structure is a limiting groove, and the limiting groove is formed on the inner side wall of the air duct in an outward protrusion manner.

5. The air purifier according to claim 4, wherein
the limiting plate is adapted to the pluggable opening, a first grabbing recess is arranged on the casing, the first grabbing recess is in communication with the pluggable opening, and the limiting plate is located above the first grabbing recess; or
a second grabbing recess and a third grabbing recess are arranged on the casing, the pluggable opening is located in the second grabbing recess and the third grabbing recess, and the second grabbing recess and the third grabbing recess are in communication with the pluggable opening.

6. The air purifier according to claim 2, wherein
the limiting structure comprises a mounting frame, the mounting frame is hung in the pluggable opening, and the fragrance diffuser is arranged on the mounting frame.

7. The air purifier according to claim 6, wherein
the mounting frame comprises a plurality of frameworks, the plurality of frameworks are cross-connected to each other, at least one limiting concave portion is formed between two adjacent frameworks, and the fragrance diffuser passes through a concave opening of the limiting concave portion so as to be snap-fitted in the limiting concave portion;
limiting blocks are arranged on sides, facing each other, of two adjacent frameworks respectively, the limiting concave portions are arranged on the two limiting blocks respectively, and concave openings of the two limiting concave portions have the same opening direction; and
openings are provided on sides, facing each other, of the two limiting blocks respectively, and the fragrance diffuser passes through the two openings so as to be clamped between the two limiting blocks.

8. (canceled)

9. The air purifier according to claim 2, wherein
the limiting structure comprises a limiting chamber, the limiting chamber is located in the air duct, the fragrance diffuser sequentially passes through the pluggable opening and a first opening of the limiting chamber so as to enter the limiting chamber, an air hole is provided on a side wall of the limiting chamber, and the air duct passes through the air hole.

10. The air purifier according to claim 9, wherein
the limiting chamber comprises a supporting plate and a baffle, the baffle is connected to the supporting plate, the baffle delimits the limiting chamber in a circumferential direction of the supporting plate, and the air hole is provided on the baffle.

11. The air purifier according to claim 9, wherein
a second opening is provided on the limiting chamber and faces the mounting frame, the air duct passes through the second opening, and the fragrance diffuser is located at the second opening and is clamped between a side wall of the limiting chamber and the mounting frame;
the functional purifying member is arranged on a first side of the mounting frame, the fragrance diffuser is arranged on a second side of the mounting frame, and the first side of the mounting frame and the second side of the mounting frame are arranged opposite each other; and/or
in an air flow direction of the air duct, the fragrance diffuser is arranged on a side, facing an air outlet of the air duct, of the mounting frame.

12. (canceled)

13. The air purifier according to claim 6, wherein
a first clamping block and a limiting plate are arranged on the mounting frame, a limiting step is arranged at the pluggable opening, and a second clamping block is arranged on an inner side wall of the air duct;
when the mounting frame moves towards the pluggable opening and passes through the pluggable opening, coordinated abutting is performed after the first clamping block passes the second clamping block, and the limiting plate abuts against the limiting step; and
when the mounting frame passes through the pluggable opening and moves away from the pluggable opening, the first clamping block passes the second clamping block and moves away from the second clamping block.

14. The air purifier according to claim 2, wherein
power of the second fan is higher than power of the first fan; and/or
the first air duct and the second air duct each correspond to at least one pluggable opening; and/or
the fragrance diffuser is located in the first air duct.

15. (canceled)

16. The air purifier according to claim 1, wherein the casing comprises:
an outer casing, wherein the air inlet, the second air outlet and the first air outlet are provided on the outer casing, and the second air duct and the first air duct are arranged in the outer casing;
a second inner casing, wherein the second air duct penetrates the second inner casing; and
a first inner casing, wherein the first air duct penetrates the first inner casing.

17. (canceled)

18. The air purifier according to claim 16, wherein a first communication port is provided on the second inner casing, a first air duct segment is arranged on the second air duct and is located between the air inlet and the first communication port, the first air duct segment is arranged around a periphery of the second inner casing, a second air duct segment is arranged on the second air duct and is located between the first communication port and the second air outlet, and the second air duct segment penetrates the second inner casing; and/or
a second communication port is provided on the first inner casing, a third air duct segment is arranged on the first air duct and is located between the air inlet and the second communication port, the third air duct segment is arranged around a periphery of the first inner casing, a fourth air duct segment is arranged on the first air duct and is located between the second communication port and the first air outlet, and the fourth air duct segment penetrates the first inner casing.

19. The air purifier according to claim 16, wherein two air inlets are provided on the outer casing, the second inner casing and the first inner casing are arranged between the two air inlets, and two first air duct segments are correspondingly formed between the outer casing and the second inner casing.

20. The air purifier according to claim 16, further comprising an air suction assembly and a functional purifying member, wherein the air suction assembly and the functional purifying member are arranged in the outer casing,
the functional purifying member comprises a plasma assembly, and the plasma assembly is arranged in the second air duct or the first air duct; and/or
the functional purifying member comprises an anion assembly, the anion assembly is arranged in the second air duct or the first air duct, the anion assembly comprises an anion generator and an anion high-voltage member, and the anion generator is connected to the anion high-voltage member; and/or
the functional purifying member comprises ultraviolet C (UVC), and the UVC is arranged in the second air duct or the first air duct.

21. (canceled)

22. The air purifier according to claim 2116, wherein
the plasma assembly comprises a high-energy plasma tube and a plasma high-voltage pack, and the plasma high-voltage pack is connected to the high-energy plasma tube; and/or
the plasma assembly comprises a low-energy ion generator and a low-energy plasma high-voltage pack, and the low-energy ion generator is connected to the low-energy plasma high-voltage pack;
the high-energy plasma tube or the low-energy ion generator is arranged in the first air duct; and in an air flow direction of the first air duct, the first fan is arranged between the high-energy plasma tube or the low-energy ion generator and the first air outlet.

23. (canceled)

24. The air purifier according to claim 16, wherein
in a radial direction of the second fan, the first fan is arranged between the second fan and the first air outlet, and the radial direction of the second fan is perpendicular to an axis of a rotation center of the second fan; and/or
the first fan is a centrifugal fan; and/or
in the radial direction of the second fan, an air outlet of the second fan and the second air outlet are sequentially arranged, and the radial direction of the second fan is perpendicular to the axis of the rotation center of the second fan; and/or
the second air outlet is provided farther away from a fixed base of the outer casing than the first air outlet; and/or
the second fan is a series excitation fan; and/or
a fan base is arranged in the outer casing, a through hole is provided on the fan base, the second fan is arranged on the fan base, and the through hole is in communication with a fan air inlet of the second fan; a ventilation cavity is provided between the fan base and a mounting surface, opposite the fan base, of the outer casing, and the second air duct sequentially passes through the ventilation cavity, the through hole, the second fan and the second air outlet; and/or
in an air flow direction of the second air duct, a width of the second air duct closer to the second air outlet is gradually reduced; and/or
the first air outlet comprises a plurality of air outlet holes, and the plurality of air outlet holes are arranged in a circle; and/or
a heater is arranged in the second air duct or the first air duct; and/or
an infrared sensor is arranged on the casing, and the infrared sensor and the second air outlet are located on the same side of the casing.

25. (canceled)

26. (canceled)

27. The air purifier according to claim 16, wherein
the second fan and the first inner casing are arranged in a preset direction, the preset direction is perpendicular to an axial direction of the second fan;
the axial direction of the second fan extends along a thickness direction of the outer casing;
at least part of the first air duct and at least part of the second air duct are arranged in the thickness direction of the outer casing and parallel.

28. The air purifier according to claim 16, wherein
a fan base is arranged in the outer casing, a through hole is provided on the fan base, the second fan is arranged on the fan base, a ventilation cavity is provided between the fan base and a mounting surface of the outer casing;
the second fan comprises a fan air inlet, a fan ventilation channel and a fan air outlet, the fan air inlet and the fan air outlet are in communication with the fan ventilation channel, the fan ventilation channel extends in a rotation axis of the second fan, the fan air inlet is located at a first axis end of the fan ventilation channel, and the fan cover is arranged at a second axis end of the fan ventilation channel, the fan air outlet is located in a radial direction of the fan ventilation channel, and the through hole is in communication with the fan ventilation channel, the second air duct segment sequentially passes through the ventilation cavity, the through hole, the fan air inlet, the fan ventilation channel, the air outlet and the second air outlet.
